(19) United States
(12) Patent Application Publication
(10) Pub. No.: US 2024/0066020 A1
(43) Pub. Date: Feb. 29, 2024

(54) COMPOSITIONS AND METHODS TO TREAT NON-ALCOHOLIC FATTY LIVER DISEASES (NAFLD)

(71) Applicant: Coherus Biosciences, Inc., Redwood City, CA (US)

(72) Inventor: Abid A. Rahman, Foster City, CA (US)

(21) Appl. No.: 17/768,538

(22) PCT Filed: Oct. 14, 2020
(86) PCT No.: PCT/US2020/055567

§ 371 Date: Apr. 13, 2022

Related U.S. Application Data
(60) Provisional application No. 62/915,101, filed on Oct. 15, 2019.

(51) Int. Cl. A61K 31/47; A61K 31/4985; A61K 31/7048; A61P 1/16

(52) U.S. Cl. CPC A61K 31/47; A61K 31/4985; A61K 31/7048; A61P 1/16

(57) ABSTRACT

Provided herein are methods and combination therapies useful for the treatment of non-alcoholic fatty liver diseases (NAFLD). In particular, provided herein are methods and combination therapies for treating NAFLD by administering a combination therapy comprising (a) the compound of Formula (I), or a pharmaceutically acceptable salt thereof, and (b) an SGLT-2 inhibitor, or a pharmaceutically acceptable salt or solvate thereof, and (c) a DPP-4 inhibitor, or a pharmaceutically acceptable salt thereof. Also provided are pharmaceutical compositions and pharmaceutical combinations comprising (a) the compound of Formula (I), or a pharmaceutically acceptable salt thereof, and (b) an SGLT-2 inhibitor, or a pharmaceutically acceptable salt or solvate thereof, and (c) a DPP-4 inhibitor, or a pharmaceutically acceptable salt thereof.

### TECHNICAL FIELD

The present disclosure relates to methods and combination therapies useful for the treatment of non-alcoholic fatty liver diseases (NAFLD). In particular, this disclosure relates to methods and combination therapies for treating NAFLD by administering a combination therapy comprising a PPARγ inhibitor that is the compound of Formula (I), or a pharmaceutically acceptable salt thereof, and an SGLT-2 inhibitor, or a pharmaceutically acceptable salt or solvate thereof, and a DPP-4 inhibitor, or a pharmaceutically acceptable salt thereof.

### BACKGROUND

Peroxisome proliferator-activated receptor γ (“PPARγ”) is one member of the nuclear receptor superfamily of ligand-activated transcription factors and has been shown to be expressed in an adipose tissue-specific manner. Its expression is induced early during the course of differentiation of several preadipocyte cell lines. Additional research has now demonstrated that PPARγ plays a pivotal role in the adipogenic signaling cascade, and also regulates the ob/leptin gene which is involved in regulating energy homeostasis and adipocyte differentiation.

Inhibitors of dipeptidyl peptidase 4 (DPP-4 inhibitors or gliptins) are a class of oral hypoglycemics that block the activity of the enzyme DPP-4. The first agent of the class—sitagliptin—was approved by the FDA in 2006. DPP-4 inhibitors increase incretin levels (GLP-1 and GIP),which inhibits glucagon release, which in turn increases insulin secretion, decreases gastric emptying, and decreases blood glucose levels.

Non-alcoholic fatty liver disease (NAFLD) is characterized by the presence of hepatic fat accumulation in the absence of secondary causes of hepatic steatosis including excessive alcohol consumption, other known liver diseases, or long-term use of a steatogenic medication (Perumpail et al., World J Gastroenterol. 2017, 23(47):8263-8438 and Chalasani et al., Hepatology. 2018, 67(1):328-357). NAFLD encompasses two categories: simple non-alcoholic fatty liver (NAFL) and non-alcoholic steatohepatitis (NASH). Typically, NAFL has a more indolent course of progression whereas NASH is a more severe form associated with inflammation that may progress more rapidly to end-stage liver disease. NAFL and/or NASH may also include scarring of the liver known as liver fibrosis or in a more severe form, liver cirrhosis. Scarring of the liver reduces liver function up to and including liver failure.

NAFLD is currently the most common liver disease in the world (Perumpail et al., World J Gastroenterol. 2017, 23(47):8263-8438) with approximately one-fourth of the adult population suffering from NAFLD worldwide (Sumida, et al., J Gastroenterol. 2018, 53:362-376). There are many risk factors associated with NAFLD including hypertension, obesity, diabetes, and hyperlipidemia with a particularly close association with type II diabetes mellitus and NAFLD (Vernon et al., Aliment Pharmacol Ther. 2011, 34:274-285).

Lifestyle interventions including dietary caloric restriction and exercise are the most effective methods of prevention and treatment for NAFLD (Sumida, et al., J Gastroenterol. 2018, 53:362-376). However, these can be difficult treatments to follow. Thus, there is a need for pharmaceuticals to treat NAFLD. Current pharmaceutical treatments that have been proposed or tested in prior trials, although are not yet approved for NAFLD include vitamin E, ω3 fatty acid, statin, metformin, orlistat, thiazolidinediones (“TZDs”), urodeoxycholic acid, pioglitazone, and pentoxifilline (Sumida, et al., J Gastroenterol. 2018, 53:362-376). However, there is currently no approved pharmacotherapy for NAFLD.

### SUMMARY

Provided herein in some embodiments is a method of treating non-alcoholic fatty liver disease (NAFLD) in a subject in need thereof comprising administering to the subject
(a) the compound of Formula (I),

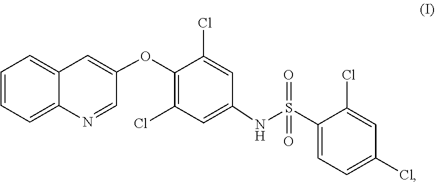 [SMILES: O=S(=O)(Nc1cc(Cl)c(Oc2cnc3ccccc3c2)c(Cl)c1)c1ccc(Cl)cc1Cl]

or a pharmaceutically acceptable salt thereof,
(b) an SGLT inhibitor, or a pharmaceutically acceptable salt or solvate thereof, and
(c) a DPP-4 inhibitor, or a pharmaceutically acceptable salt thereof;
   wherein the amounts of (a), (b), and (c) together are effective in treating NAFLD.

Provided herein in some embodiments is a method of treating a subject, the method comprising:
selecting a subject having non-alcoholic fatty liver disease (NAFLD); and
administering
(a) the compound of Formula (I),

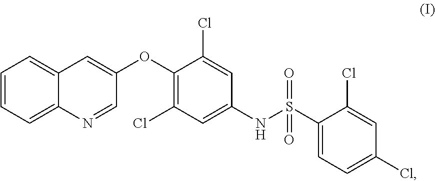 [SMILES: O=S(=O)(Nc1cc(Cl)c(Oc2cnc3ccccc3c2)c(Cl)c1)c1ccc(Cl)cc1Cl]

or a pharmaceutically acceptable salt thereof,
(b) an SGLT inhibitor, or a pharmaceutically acceptable salt or solvate thereof, and
(c) a DPP-4 inhibitor, or a pharmaceutically acceptable salt thereof;
   wherein the amounts of (a), (b), and (c) together are effective in treating NAFLD.

Provided herein in some embodiments is a method of treating a subject, the method comprising:
identifying a subject having non-alcoholic fatty liver disease (NAFLD); and
administering
(a) the compound of Formula (I),

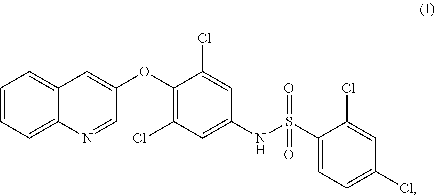 [SMILES: O=S(=O)(Nc1cc(Cl)c(Oc2cnc3ccccc3c2)c(Cl)c1)c1ccc(Cl)cc1Cl]

or a pharmaceutically acceptable salt thereof,
(b) an SGLT inhibitor, or a pharmaceutically acceptable salt or solvate thereof, and
(c) a DPP-4 inhibitor, or a pharmaceutically acceptable salt thereof;
   wherein the amounts of (a), (b), and (c) together are effective in treating NAFLD.

Provided herein in some embodiments is a method of treating non-alcoholic fatty liver disease (NAFLD) in a subject in need thereof comprising administering to the subject
(a) a therapeutically effective amount of the compound of Formula (I),

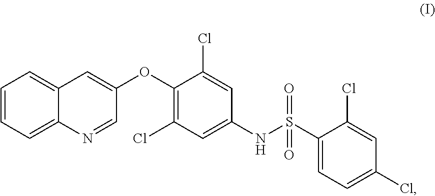 [SMILES: O=S(=O)(Nc1cc(Cl)c(Oc2cnc3ccccc3c2)c(Cl)c1)c1ccc(Cl)cc1Cl]

or a pharmaceutically acceptable salt thereof, and
(b) a therapeutically effective amount of an SGLT inhibitor, or a pharmaceutically acceptable salt or solvate thereof, and
(c) a therapeutically effective amount of a DPP-4 inhibitor, or a pharmaceutically acceptable salt thereof.

Provided herein in some embodiments is a method of treating a subject, the method comprising:
selecting a subject having non-alcoholic fatty liver disease (NAFLD); and
administering
(a) a therapeutically effective amount of the compound of Formula (I),

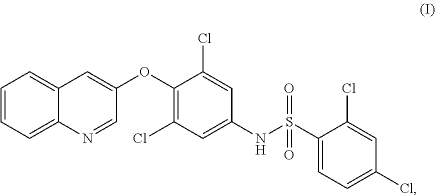 [SMILES: O=S(=O)(Nc1cc(Cl)c(Oc2cnc3ccccc3c2)c(Cl)c1)c1ccc(Cl)cc1Cl]

or a pharmaceutically acceptable salt thereof, and
(b) a therapeutically effective amount of an SGLT inhibitor, or a pharmaceutically acceptable salt or solvate thereof, and
(c) a therapeutically effective amount of a DPP-4 inhibitor, or a pharmaceutically acceptable salt thereof, to the selected subject.

Provided herein in some embodiments is a method of treating fibrosis in a subject in need thereof comprising administering to the subject
(a) the compound of Formula (I),

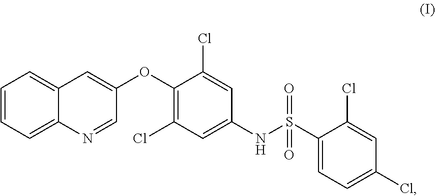 [SMILES: O=S(=O)(Nc1cc(Cl)c(Oc2cnc3ccccc3c2)c(Cl)c1)c1ccc(Cl)cc1Cl]

or a pharmaceutically acceptable salt thereof,
(b) an SGLT inhibitor, or a pharmaceutically acceptable salt or solvate thereof, and
(c) a DPP-4 inhibitor, or a pharmaceutically acceptable salt thereof;
   wherein the amounts of (a), (b), and (c) together are effective in treating fibrosis.

Provided herein in some embodiments is a method of treating fibrosis in a subject in need thereof comprising administering to the subject
(a) a therapeutically effective amount of the compound of Formula (I),

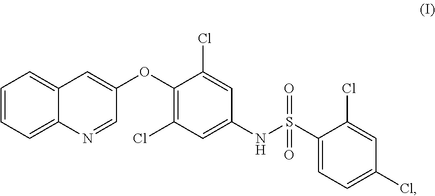 [SMILES: O=S(=O)(Nc1cc(Cl)c(Oc2cnc3ccccc3c2)c(Cl)c1)c1ccc(Cl)cc1Cl]

or a pharmaceutically acceptable salt thereof,
(b) a therapeutically effective amount of an SGLT inhibitor, or a pharmaceutically acceptable salt or solvate thereof, and
(c) a therapeutically effective amount of a DPP-4 inhibitor, or a pharmaceutically acceptable salt thereof.

Provided herein in some embodiments is a method of treating a subject, the method comprising:
selecting a subject having non-alcoholic fatty liver disease (NAFLD); and
administering
(a) the compound of Formula (I),

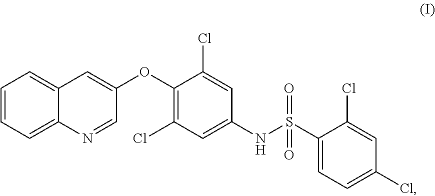 [SMILES: O=S(=O)(Nc1cc(Cl)c(Oc2cnc3ccccc3c2)c(Cl)c1)c1ccc(Cl)cc1Cl]

or a pharmaceutically acceptable salt thereof,
(b) an SGLT inhibitor, or a pharmaceutically acceptable salt or solvate thereof, and
(c) a DPP-4 inhibitor, or a pharmaceutically acceptable salt thereof, to the selected subject;
   wherein the amounts of (a), (b), and (c) together are effective in treating NAFLD.

Provided herein in some embodiments is a method of treating a subject, the method comprising:
identifying a subject having non-alcoholic fatty liver disease (NAFLD); and
administering
(a) the compound of Formula (I),

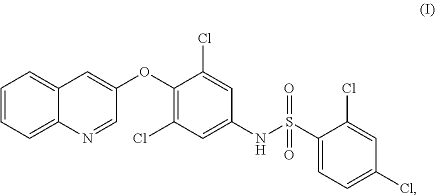 [SMILES: O=S(=O)(Nc1cc(Cl)c(Oc2cnc3ccccc3c2)c(Cl)c1)c1ccc(Cl)cc1Cl]

or a pharmaceutically acceptable salt thereof,
(b) an SGLT inhibitor, or a pharmaceutically acceptable salt or solvate thereof, and
(c) a DPP-4 inhibitor, or a pharmaceutically acceptable salt thereof, to the subject;
   wherein the amounts of (a), (b), and (c) together are effective in treating NAFLD.

Provided herein in some embodiments is a method of treating non-alcoholic fatty liver disease (NAFLD) in a subject in need thereof comprising administering to the subject
(a) a therapeutically effective amount of the compound of Formula (I),

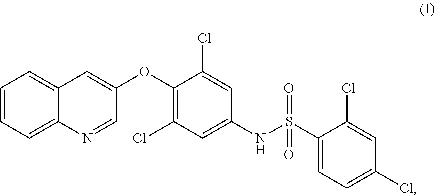 [SMILES: O=S(=O)(Nc1cc(Cl)c(Oc2cnc3ccccc3c2)c(Cl)c1)c1ccc(Cl)cc1Cl]

or a pharmaceutically acceptable salt thereof,
(b) a therapeutically effective amount of an SGLT inhibitor, or a pharmaceutically acceptable salt or solvate thereof, and
(c) a therapeutically effective amount of a DPP-4 inhibitor, or a pharmaceutically acceptable salt thereof.

Provided herein in some embodiments is a method of treating a subject, the method comprising:
selecting a subject having non-alcoholic fatty liver disease (NAFLD); and
administering
(a) a therapeutically effective amount of the compound of Formula (I),

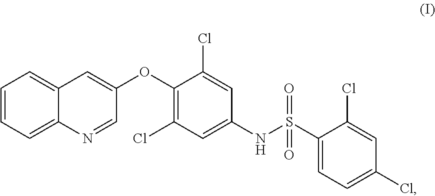 [SMILES: O=S(=O)(Nc1cc(Cl)c(Oc2cnc3ccccc3c2)c(Cl)c1)c1ccc(Cl)cc1Cl]

or a pharmaceutically acceptable salt thereof,
(b) a therapeutically effective amount of an SGLT inhibitor, or a pharmaceutically acceptable salt or solvate thereof, and
(c) a therapeutically effective amount of a DPP-4 inhibitor, or a pharmaceutically acceptable salt thereof, to the selected subject.

Provided herein in some embodiments is a method of treating fibrosis in a subject in need thereof comprising administering to the subject
(a) a therapeutically effective amount of the compound of Formula (I),

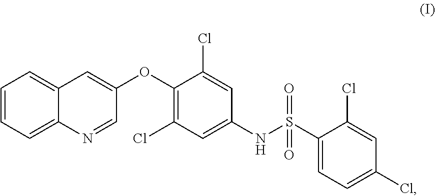 [SMILES: O=S(=O)(Nc1cc(Cl)c(Oc2cnc3ccccc3c2)c(Cl)c1)c1ccc(Cl)cc1Cl]

or a pharmaceutically acceptable salt thereof,
(b) a therapeutically effective amount of an SGLT inhibitor, or a pharmaceutically acceptable salt or solvate thereof, and
(c) a therapeutically effective amount of a DPP-4 inhibitor, or a pharmaceutically acceptable salt thereof;
   wherein the amounts of (a), (b), and (c) together are effective in treating fibrosis.

Provided herein in some embodiments is a method of treating fibrosis in a subject in need thereof comprising administering to the subject
(a) a therapeutically effective amount of the compound of Formula (I),

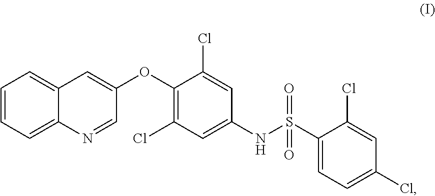 [SMILES: O=S(=O)(Nc1cc(Cl)c(Oc2cnc3ccccc3c2)c(Cl)c1)c1ccc(Cl)cc1Cl]

or a pharmaceutically acceptable salt thereof,
(b) a therapeutically effective amount of an SGLT inhibitor, or a pharmaceutically acceptable salt or solvate thereof, and
(c) a therapeutically effective amount of a DPP-4 inhibitor, or a pharmaceutically acceptable salt thereof.

In some embodiments, (a), (b), and (c) are administered concurrently. In other embodiments, (a), (b), and (c) are administered sequentially in any order.

Provided herein in some embodiments is a pharmaceutical composition comprising
(a) the compound of Formula (I),

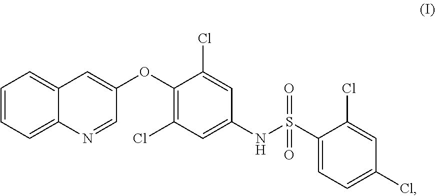 [SMILES: O=S(=O)(Nc1cc(Cl)c(Oc2cnc3ccccc3c2)c(Cl)c1)c1ccc(Cl)cc1Cl]

or a pharmaceutically acceptable salt thereof,
(b) an SGLT inhibitor, or a pharmaceutically acceptable salt or solvate thereof,
(c) a DPP-4 inhibitor, or a pharmaceutically acceptable salt thereof, and one or more pharmaceutical excipients;
   wherein the amounts of (a), (b), and (c) together are effective in treating NAFLD.

In some embodiments of the pharmaceutical compositions provided herein, the pharmaceutical compositions comprise at least one pharmaceutically acceptable carrier.

In some more particular embodiments, a method as provided herein comprises administering a pharmaceutical composition as provided herein to a subject twice a day, daily, every other day, three times a week, twice a week, weekly, every other week, twice a month, or monthly.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DETAILED DESCRIPTION

### Definitions

Reference to the term “about” has its usual meaning in the context of pharmaceutical compositions to allow for reasonable variations in amounts that can achieve the same effect and also refers herein to a value of plus or minus 10% of the provided value. For example, “about 20” means or includes amounts from 18 to and including 22.

The term “administration” or “administering” refers to a method of giving a dosage of a compound or pharmaceutical composition to a vertebrate or invertebrate, including a mammal, a bird, a fish, or an amphibian. The preferred method of administration can vary depending on various factors, e.g., the components of the pharmaceutical composition, the site of the disease, and the severity of the disease.

The term “CHS-131” as used herein refers to a compound of Formula (I):

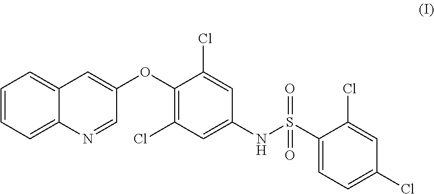 [SMILES: O=S(=O)(Nc1cc(Cl)c(Oc2cnc3ccccc3c2)c(Cl)c1)c1ccc(Cl)cc1Cl]

or a pharmaceutically acceptable salt thereof.
The compound of Formula (I) is a selective peroxisome proliferator-activated receptor (PPAR) γ modulator. The compound of Formula (I) is disclosed in, for example, U.S. Pat. Nos. 7,041,691; 6,200,995; 6,583,157; 6,653,332; and U.S. Publication Application No. 2016/0260398, the contents of each of which are incorporated by reference herein in their entireties.

The compound of Formula (I) can be prepared, for example, by the methods described in U.S. Pat. Nos. 6,583,157 or 6,200,995, each of which is incorporated by reference in its entirety herein. In some embodiments, different salts, e.g., besylate, tosylate HCl, or HBr salts, and/or polymorphs of the compound of Formula (I) are used within the methods and compositions described herein. Salts and polymorphs of the compound of Formula (I), such as those provided herein, can be prepared according to the methods described in U.S. Pat. Nos. 6,583,157 and 7,223,761, the contents of each of which are incorporated by reference in their entireties.

The term “SGLT inhibitor” as used herein refers to a compound that inhibits one or more Sodium Glucose Co-Transporters. In one embodiment, an SLGT inhibitor is a compound that inhibits the Sodium Glucose Co-Transporter-1 (SGLT-1). In another embodiment, an SLGT inhibitor is a compound that inhibits the Sodium Glucose Co-Transporter-2 (SGLT-2). In yet another embodiment, an SLGT inhibitor is a compound that inhibits both SGLT-1 and SGLT-2.

The term “SGLT-1 inhibitor” as used herein refers to a compound that inhibits the Sodium Glucose Co-Transporter-1 (SGLT-1). SGLT-1 primarily absorbs glucose in the small intestine and also reabsorbs glucose in the kidneys. By disrupting these functions, SGLT-1 inhibitors exert a glucose-lowering effect. See, Spatola et al., Diabetes Ther. 2017; 9(1):427-430. The term “SGLT-1 inhibitor” is not limited to compounds that only inhibit SGLT-1, thus includes compounds that have other activities in addition to SGLT-1 inhibition. Examples of SGLT-1 inhibitors include, but are not limited to, LX2761 (Lexicon Pharmaceuticals; See, Powell et al., J Pharmacol Exp Ther. 2017 July; 362(1):85-97), licofliglozin and sotagliflozin (ZYNQUISTA™).

The term “SGLT-2 inhibitor” as used herein refers to a compound that inhibits the Sodium Glucose Co-Transporter-2 (SGLT-2). SGLT-2 inhibitors disrupt reabsorption of glucose by the kidneys and thus exert a glucose-lowering effect. By enhancing glucosuria, independently of insulin, SLGT-2 inhibitors have been shown to treat type 2 diabetes and improve cardiovascular outcomes. See, Wright, 2001, Am J Physiol Renal Physiol 280:F10; and Scheen, 2018, Circ Res 122:1439. SGLT2 inhibitors include a class of drugs known as gliflozins. The term “SGLT-2 inhibitor” is not limited to compounds that only inhibit SGLT-2, thus includes compounds that have other activities in addition to SGLT-2 inhibition. Examples of SGLT-2 inhibitors include, but are not limited to, bexagliflozin, canagliflozin (INVOKANA®), dapagliflozin (FARXIGA®), empagliflozin (JARDIANCE®), ertugliflozin (STEGLATRO™), ipragliflozin (SUGLAT®), luseogliflozin (LUSEFI®), remogliflozin, serfliflozin, licofliglozin, sotagliflozin (ZYNQUISTA™), and tofogliflozin.

The term “SGLT-1/2 dual inhibitor” and “SGLT dual inhibitor” as used herein refers to a compound that inhibits both SGLT-1 and SGLT-2. See, Danne, et al., Diabetes Technol Ther. 2018 June; 20(S2):S269-S277. Examples of dual inhibitors include, but are not limited to, licofliglozin and sotagliflozin (ZYNQUISTA™).

The term “DPP-4 inhibitor” as used herein refers to an inhibitor of the dipeptidyl peptidase 4 enzyme. Examples of DPP-4 inhibitors include, but are not limited to, sitagliptin, vildagliptin, saxagliptin, linagliptin, gemigliptin, anagliptin, teneligliptin, alogliptin, trelagliptin, omarigliptin, evogliptin, and dutogliptin.

A “therapeutically effective amount” of a compound as provided herein is an amount that is sufficient to achieve the desired therapeutic effect and can vary according to the nature and severity of the disease condition, and the potency of the compound. A therapeutic effect is the relief, to some extent, of one or more of the symptoms of the disease, and can include curing a disease. “Curing” means that the symptoms of active disease are eliminated. However, certain long-term or permanent effects of the disease can exist even after a cure is obtained (such as, e.g., extensive tissue damage). In some embodiments, a “therapeutically effective amount” of a compound as provided herein refers to an amount of the compound that is effective as a monotherapy. In some embodiments, the therapeutic effect is determined from one or more parameters selected from the NAFLD Activity Score (NAS), hepatic steatosis, hepatic inflammation, biomarkers indicative of liver damage, and liver fibrosis and/or liver cirrhosis. For example, a therapeutic effect can include one or more of a decrease in symptoms, a decrease in the NAS, a reduction in the amount of hepatic steatosis, a decrease in hepatic inflammation, a decrease in the level of biomarkers indicative of liver damage, and a reduction in liver fibrosis and/or liver cirrhosis, a lack of further progression of liver fibrosis and/or liver cirrhosis, or a slowing of the progression of liver fibrosis and/or liver cirrhosis following administration of a compound or compounds as described herein.

In some embodiments, the amounts of the two or more compounds as provided herein together are effective in treating NAFLD (e.g., the amounts of the compound of Formula (I), an SGLT-2 inhibitor, and a DPP-4 inhibitor, together are effective in treating NAFLD). In such embodiments, the amount of each agent is also referred to as a “jointly therapeutically effective amount.” For example, the therapeutic agents of a combination described herein are given to the subject simultaneously or separately (e.g., in a chronologically staggered manner, for example a sequence-specific manner) in such time intervals that they show an interaction (e.g., a joint therapeutic effect). For example, wherein the amounts of (a) the compound of Formula (I), or a pharmaceutically acceptable salt thereof, (b) an SGLT-2 inhibitor, or a pharmaceutically acceptable salt or solvate thereof, and (c) a DPP-4 inhibitor, or a pharmaceutically acceptable salt thereof, together are effective in treating NAFLD, the joint therapeutic effect of the combination of (a) the compound of Formula (I), or a pharmaceutically acceptable salt thereof, (b) an SGLT-2 inhibitor, or a pharmaceutically acceptable salt or solvate thereof, and (c) a DPP-4 inhibitor, or a pharmaceutically acceptable salt thereof, is 10%-100% greater than, such as 10%-50%, 20%-60%, 30%-70%, 40%-80%, 50%-90%, or 60%-100%, greater than, such as 10%, 20%, 30%, 40%, 50%, 60%, 70%, 80%, or 90% greater than, the therapeutic effect of the compound of Formula (I), or a pharmaceutically acceptable salt, prodrug, or isomer thereof alone. In some embodiments, wherein the amounts of (a) the compound of Formula (I), or a pharmaceutically acceptable salt thereof, (b) an SGLT-2 inhibitor, or a pharmaceutically acceptable salt or solvate thereof, and (c) a DPP-4 inhibitor, or a pharmaceutically acceptable salt thereof, together are effective in treating NAFLD, the joint therapeutic effect of the combination of (a) the compound of Formula (I), or a pharmaceutically acceptable salt thereof, (b) an SGLT-2 inhibitor, or a pharmaceutically acceptable salt or solvate thereof, and (c) a DPP-4 inhibitor, or a pharmaceutically acceptable salt thereof, is 10%-100% greater than, such as 10%-50%, 20%-60%, 30%-70%, 40%-80%, 50%-90%, or 60%-100%, greater than, such as 10%, 20%, 30%, 40%, 50%, 60%, 70%, 80%, or 90% greater than, the therapeutic effect of the SGLT-2 inhibitor alone, or the pharmaceutically acceptable salt or solvate thereof, or the DPP-4 inhibitor alone, or the pharmaceutically acceptable salt thereof.

The term “preventing” as used herein means the prevention of the onset, recurrence or spread, in whole or in part, of the disease or condition as described herein, or a symptom thereof.

As used herein, the terms “treat” or “treatment” refer to therapeutic or palliative measures. Beneficial or desired clinical results include, but are not limited to, alleviation, in whole or in part, of symptoms associated with a disease or disorder or condition, diminishment of the extent of disease, stabilized (i.e., not worsening) state of disease, delay or slowing of disease progression, amelioration or palliation of the disease state (e.g., one or more symptoms of the disease), and remission (whether partial or total), whether detectable or undetectable. “Treatment” can also mean prolonging survival as compared to expected survival if not receiving treatment.

As used herein, “subject” refers to any subject, particularly a mammalian subject, for whom diagnosis, prognosis, or therapy is desired, for example, a human.

The terms “treatment regimen” and “dosing regimen” are used interchangeably to refer to the dose and timing of administration of each therapeutic agent in a combination of the invention.

The term “pharmaceutical combination,” as used herein, refers to a pharmaceutical treatment resulting from the mixing or combining of more than one active ingredient and includes both fixed and non-fixed combinations of the active ingredients.

The term “combination therapy” as used herein refers to a dosing regimen of two different therapeutically active agents (i.e., the components or combination partners of the combination) (e.g., the compound of Formula (I), or a pharmaceutically acceptable salt thereof, an SGLT-2 inhibitor, or a pharmaceutically acceptable salt or solvate thereof, and a DPP-4 inhibitor, or a pharmaceutically acceptable salt thereof), wherein the therapeutically active agents are administered together or separately in a manner prescribed by a medical care taker or according to a regulatory agency as defined herein. In some embodiments, a combination therapy comprises a combination of (a) the compound of Formula (I), or a pharmaceutically acceptable salt thereof, (b) SGLT-2 inhibitor (e.g., empagliflozin or dapagliflozin), or a pharmaceutically acceptable salt or solvate thereof, and (c) a DPP-4 inhibitor (e.g., sitagliptin), or a pharmaceutically acceptable salt thereof. In some embodiments, a combination therapy consists essentially of a combination of (a) the compound of Formula (I), or a pharmaceutically acceptable salt thereof, (b) SGLT-2 inhibitor (e.g., empagliflozin or dapagliflozin), or a pharmaceutically acceptable salt or solvate thereof, and (c) a DPP-4 inhibitor (e.g., sitagliptin), or a pharmaceutically acceptable salt thereof.

The term “fixed combination” means that the compound of Formula (I), or a pharmaceutically acceptable salt thereof, the SGLT-2 inhibitor, or a pharmaceutically acceptable salt or solvate thereof, and the DPP-4 inhibitor, or a pharmaceutically acceptable salt thereof, are each administered to a subject simultaneously in the form of a single composition or dosage.

The term “non-fixed combination” means that the compound of Formula (I), or a pharmaceutically acceptable salt thereof, the SGLT-2 inhibitor, or a pharmaceutically acceptable salt or solvate thereof, and the DPP-4 inhibitor, or a pharmaceutically acceptable salt thereof, are formulated as separate compositions or dosages such that they may be administered to a subject in need thereof concurrently or sequentially with variable intervening time limits, wherein such administration provides effective levels of the three compounds in the body of the subject. These also apply to cocktail therapies, e.g. the administration of three or more active ingredients.

As can be appreciated in the art, a combination therapy can be administered to a subject for a period of time. In some embodiments, the period of time occurs following the administration of a different therapeutic treatment/agent or a different combination of therapeutic treatments/agents to the subject. In some embodiments, the period of time occurs before the administration of a different therapeutic treatment/agent or a different combination of therapeutic treatments/agents to the subject.

A suitable period of time can be determined by one skilled in the art (e.g., a physician). As can be appreciated in the art, a suitable period of time can be determined by one skilled in the art based on one or more of: the stage of disease in the subject, the mass and sex of the subject, clinical trial guidelines (e.g., those on the fda.gov website), and information on the approved drug label. For example a suitable period of time can be, e.g., from 1 week to 2 years, 1 week to 22 months, 1 week to 20 months, 1 week to 18 months, 1 week to 16 months, 1 week to 14 months, 1 week to 12 months, 1 week to 10 months, 1 week to 8 months, 1 week to 6 months, 1 week to 4 months 1 week to 2 months, 1 week to 1 month, 2 weeks to 2 years, 2 weeks to 22 months, 2 weeks to 20 months, 2 weeks to 18 months, 2 weeks to 16 months, 2 weeks to 14 months, 2 weeks to 12 months, 2 weeks to 10 months, 2 weeks to 8 months, 2 weeks to 6 months, 2 weeks to 4 months, 2 weeks to 2 months, 2 weeks to 1 month, 1 month to 2 years, 1 month to 22 months, 1 month to 20 months, 1 month to 18 months, 1 month to 16 months, 1 month to 14 months, 1 month to 12 months, 1 month to 10 months, 1 month to 8 months, 1 month to 6 months, 1 month to 4 months, 1 month to 2 months, 2 months to 2 years, 2 months to 22 months, 2 months to 20 months, 2 months to 18 months, 2 months to 16 months, 2 months to 14 months, 2 months to 12 months, 2 months to 10 months, 2 months to 8 months, 2 months to 6 months, 2 months to 4 months, 3 months to 2 years, 3 months to 22 months, 3 months to 20 months, 3 months to 18 months, 3 months to 16 months, 3 months to 14 months, 3 months to 12 months, 3 months to 10 months, 3 months to 8 months, 3 months to 6 months, 4 months to 2 years, 4 months to 22 months, 4 months to 20 months, 4 months to 18 months, 4 months to 16 months, 4 months to 14 months, 4 months to 12 months, 4 months to 10 months, 4 months to 8 months, 4 months to 6 months, 6 months to 2 years, 6 months to 22 months, 6 months to 20 months, 6 months to 18 months, 6 months to 16 months, 6 months to 14 months, 6 months to 12 months, 6 months to 10 months, 6 months to 8 months, 8 months to 2 years, 8 months to 22 months, 8 months to 20 months, 8 months to 18 months, 8 months to 16 months, 8 months to 14 months, 8 months to 12 months, 8 months to 10 months, 10 months to 2 years, 10 months to 22 months, 10 months to 20 months, 10 months to 18 months, 10 months to 16 months, 10 months to 14 months, 10 months to 12 months, 12 months to 2 years, 12 months to 22 months, 12 months to 20 months, 12 months to 18 months, 12 months to 16 months, or 12 months to 14 months, inclusive. In some embodiments, a suitable period of time can be, e.g., from 1 month to 10 years, 1 month to 5 years, 5 years to 10 years, 3 years to 7 years, 1 year to 3 years, 3 years to 6 years, 6 years to 9 years, 2 years to 3 years, 3 years to 4 years, 4 years to 5 years, 5 years to 6 years, 6 years to 7 years, 7 years to 8 years, 8 years to 9 years, or 9 years to 10 years.

The phrases “prior to a period of time” or “before a period of time” refer to (1) the completion of administration of treatment to the subject before the first administration of a therapeutic agent during the period of time, and/or (2) the administration of one or more therapeutic agents to the subject before a first administration of a therapeutic agent in the combination therapy described herein during the period of time, such that the one or more therapeutic agents are present in subtherapeutic and/or undetectable levels in the subject at the time the first administration of a therapeutic agent in the combination therapy is performed during the period of time. In some embodiments, the phrase “prior to a period of time” or “before a period of time” refer to the administration of one or more therapeutic agents to the subject before a first administration of a therapeutic agent in the combination therapy during the period of time, such that the one or more therapeutic agents are present in subtherapeutic levels in the subject at the time the first administration of a therapeutic agent in the combination therapy is performed during the period of time. In some embodiments, the phrase “prior to a period of time” or “before a period of time” refer to the administration of one or more therapeutic agents to the subject before a first administration of a therapeutic agent in the combination therapy during the period of time, such that the one or more therapeutic agents are present in undetectable levels in the subject at the time the first administration of a therapeutic agent in the combination therapy is performed during the period of time. In some embodiments, the phrase “prior to a period of time” or “before a period of time” refer to the administration of one or more therapeutic agents to the subject before a first administration of a therapeutic agent in the combination therapy during the period of time, such that the one or more therapeutic agents are present in subtherapeutic and/or undetectable levels in the subject at the time the first administration of a therapeutic agent in the combination therapy is performed during the period of time.

The term “synergy” or “synergistic” is used herein to mean that the effect of the combination of the two therapeutic agents of the combination therapy is greater than the sum of the effect of each agent when administered alone. A “synergistic amount” or “synergistically effective amount” is an amount of the combination of the two combination partners that results in a synergistic effect, as “synergistic” is defined herein. Determining a synergistic interaction between two combination partners, the optimum range for the effect and absolute dose ranges of each component for the effect may be definitively measured by administration of the combination partners over different w/w (weight per weight) ratio ranges and doses to subjects in need of treatment. However, the observation of synergy in in vitro models or in vivo models can be predictive of the effect in humans and other species and in vitro models or in vivo models exist, as described herein, to measure a synergistic effect and the results of such studies can also be used to predict effective dose and plasma concentration ratio ranges and the absolute doses and plasma concentrations required in humans and other species by the application of pharmacokinetic/pharmacodynamic methods. Exemplary synergistic effects includes, but are not limited to, enhanced therapeutic efficacy, decreased dosage at equal or increased level of efficacy, reduced or delayed development of drug resistance, and simultaneous enhancement or equal therapeutic actions (e.g., the same therapeutic effect as at least one of the therapeutic agents) and reduction of unwanted drug effects (e.g. side effects and adverse events) of at least one of the therapeutic agents.

For example, a synergistic ratio of two therapeutic agents can be identified by determining a synergistic effect in, for example, an art-accepted in vivo model (e.g., an animal model) of NAFLD (e.g., the diet induced obese (DIO)-NASH mouse model or any of the models described in Herck et al. Nutrients. 2017 October; 9(10): 1072, which is incorporated by reference herein in its entirety).

In some embodiments, “synergistic effect” as used herein refers to a combination of (a) the compound of Formula (I), or a pharmaceutically acceptable salt thereof, (b) SGLT-2 inhibitor (e.g., empagliflozin or dapagliflozin), or a pharmaceutically acceptable salt or solvate thereof, and (c) a DPP-4 inhibitor (e.g., sitagliptin), or a pharmaceutically acceptable salt thereof, producing an effect, for example, any of the beneficial or desired results including clinical results as described herein, for example slowing the symptomatic progression of NAFLD, or symptoms thereof, which is greater than the sum of effect observed when (a) the compound of Formula (I), or a pharmaceutically acceptable salt thereof, (b) SGLT-2 inhibitor (e.g., empagliflozin or dapagliflozin), or a pharmaceutically acceptable salt or solvate thereof, or (c) a DPP-4 inhibitor (e.g., sitagliptin), or a pharmaceutically acceptable salt thereof are administered alone.

In some embodiments, “synergistic effect” as used herein refers to a combination of (a) the compound of Formula (I), or a pharmaceutically acceptable salt thereof, (b) SGLT-2 inhibitor (e.g., empagliflozin or dapagliflozin), or a pharmaceutically acceptable salt or solvate thereof, and (c) a DPP-4 inhibitor (e.g., sitagliptin), or a pharmaceutically acceptable salt thereof, producing an effect, for example, any of the beneficial or desired results including clinical results as described herein, for example slowing the symptomatic progression of NAFLD, or symptoms thereof, which is greater than the sum of the effect observed when the same amount of (a) the compound of Formula (I), or a pharmaceutically acceptable salt thereof, (b) SGLT-2 inhibitor (e.g., empagliflozin or dapagliflozin), or a pharmaceutically acceptable salt or solvate thereof, and (c) a DPP-4 inhibitor (e.g., sitagliptin), or a pharmaceutically acceptable salt thereof, as in the combination are administered alone.

In some embodiments, “synergistic effect” as used herein refers to a combination (a) the compound of Formula (I), or a pharmaceutically acceptable salt thereof, (b) SGLT-2 inhibitor (e.g., empagliflozin or dapagliflozin), or a pharmaceutically acceptable salt or solvate thereof, and (c) a DPP-4 inhibitor (e.g., sitagliptin), or a pharmaceutically acceptable salt thereof producing, for example, a therapeutic effect using a smaller dose of either of (a) the compound of Formula (I), or a pharmaceutically acceptable salt thereof, (b) SGLT-2 inhibitor (e.g., empagliflozin or dapagliflozin), or a pharmaceutically acceptable salt or solvate thereof, and (c) a DPP-4 inhibitor (e.g., sitagliptin), or a pharmaceutically acceptable salt thereof, compared to the amount used in monotherapy. For example, the dose of (a) the compound of Formula (I), or a pharmaceutically acceptable salt thereof, administered in combination with (b) an SGLT-2 inhibitor, or a pharmaceutically acceptable salt or solvate thereof, and (c) a DPP-4 inhibitor, or a pharmaceutically acceptable salt thereof, may be about 0.5% to about 90% of the dose of the compound of Formula (I), or pharmaceutically acceptable salt thereof, administered as a monotherapy to produce the same therapeutic effect, e.g., any of the beneficial or desired results including clinical results as described herein, for example slowing the symptomatic progression of NAFLD, or symptoms thereof. For example, the dose of (a) the compound of Formula (I), or a pharmaceutically acceptable salt thereof, administered in combination with (b) an SGLT-2 inhibitor, or a pharmaceutically acceptable salt or solvate thereof, and (c) a DPP-4 inhibitor, or a pharmaceutically acceptable salt thereof may be about 0.5% to 30%, about 30% to about 60%, about 60% to about 90%, such as about 0.5%, about 5%, about 10%, about 15%, about 20%, about 25%, about 30%, about 35%, about 40%, about 45%, about 50%, about 55%, about 60%, about 65%, about 70%, about 75%, about 80%, about 85%, or about 90% of the dose of the compound of Formula (I) administered as a monotherapy. As another example, the dose of the SGLT-2 inhibitor, or a pharmaceutically acceptable salt or solvate thereof and/or the DPP-4 inhibitor, or a pharmaceutically acceptable salt thereof, administered in combination with the compound of Formula (I), or a pharmaceutically acceptable salt thereof, may be about 0.5% to about 90% of the dose of the SGLT-2 inhibitor, or a pharmaceutically acceptable salt or solvate thereof and/or the DPP-4 inhibitor, or a pharmaceutically acceptable salt thereof administered as a monotherapy to produce the same therapeutic effect, e.g., any of the beneficial or desired results including clinical results as described herein, for example slowing the symptomatic progression of NAFLD, or symptoms thereof. For example, the dose of the SGLT-2 inhibitor, or a pharmaceutically acceptable salt or solvate thereof and/or the DPP-4 inhibitor, or a pharmaceutically acceptable salt thereof administered in combination with the compound of Formula (I), or a pharmaceutically acceptable salt thereof, may be about 0.5% to 30%, about 30% to about 60%, about 60% to about 90%, such as about 0.5%, about 5%, about 10%, about 15%, about 20%, about 25%, about 30%, about 35%, about 40%, about 45%, about 50%, about 55%, about 60%, about 65%, about 70%, about 75%, about 80%, about 85%, or about 90% of the dose of the SGLT-2 inhibitor, or a pharmaceutically acceptable salt or solvate thereof and/or the DPP-4 inhibitor, or a pharmaceutically acceptable salt thereof administered as a monotherapy.

In some embodiments, “synergistic effect” as used herein refers to a combination of (a) the compound of Formula (I), or a pharmaceutically acceptable salt thereof, (b) an SGLT-2 inhibitor, or a pharmaceutically acceptable salt or solvate thereof, and (c) a DPP-4 inhibitor, or a pharmaceutically acceptable salt thereof, producing a desired therapeutic effect and a reduction in an unwanted drug effect, side effect, or adverse event.

In some embodiments, the desired therapeutic effect is the same therapeutic effect observed in monotherapy of the compound of (a) the compound of Formula (I), or a pharmaceutically acceptable salt thereof, (b) an SGLT-2 inhibitor, or a pharmaceutically acceptable salt or solvate thereof, and (c) a DPP-4 inhibitor, or a pharmaceutically acceptable salt thereof, e.g., any of the beneficial or desired results including clinical results as described herein, for example slowing the symptomatic progression of NAFLD, or symptoms thereof.

In some embodiments, an unwanted drug effect, side effect, or adverse event is associated with or observed in monotherapy of (a) the compound of Formula (I), or a pharmaceutically acceptable salt thereof, (b) an SGLT-2 inhibitor, or a pharmaceutically acceptable salt or solvate thereof, and/or (c) a DPP-4 inhibitor, or a pharmaceutically acceptable salt thereof. In some embodiments, an unwanted drug effect, side effect, or adverse event is associated with or observed in monotherapy of the compound of Formula (I), or a pharmaceutically acceptable salt thereof includes, but is not limited to edema, weight gain, hypertension, cardiovascular disease, and cardiovascular events (e.g. cardiovascular death, nonfatal myocardial infarction and nonfatal stroke).

### Methods and Combination Therapies

The present disclosure relates to methods and combination therapies for treating non-alcoholic fatty liver disease (NAFLD) in a subject in need thereof by administering (a) the compound of Formula (I):

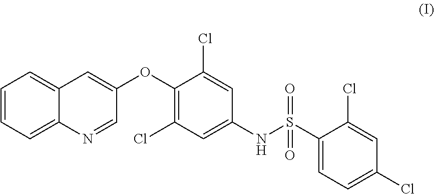 [SMILES: O=S(=O)(Nc1cc(Cl)c(Oc2cnc3ccccc3c2)c(Cl)c1)c1ccc(Cl)cc1Cl]

or a pharmaceutically acceptable salt thereof, and (b) a sodium-glucose cotransporter (SGLT) inhibitor, or a pharmaceutically acceptable salt or solvate thereof, and (c) a DPP-4 inhibitor, or a pharmaceutically acceptable salt thereof. In some embodiments, the SGLT inhibitor is a sodium-glucose cotransporter-2 (SGLT-2) inhibitor.

In some embodiments, the subject has Type I diabetes as a comorbidity. In other embodiments, the subject has Type II diabetes as a comorbidity. In some embodiments, the subject has adequate glycemic control, prior to receiving the combination of (a), (b), and (c). For example, in some embodiments, the subject has an HbA_{1c} level of ≤10%, or ≤9%, or ≤8%, or ≤7%, or ≤6%, or ≤5%, or ≤4%, or any value in between, prior to receiving the combination of (a), (b), and (c). In some embodiments, the subject has an HbA_{1c} level of about 4% to about 6%, prior to receiving the combination of (a), (b), and (c). In other embodiments, the subject has an HbA_{1c} level of about 5% to about 8%, prior to receiving the combination of (a), (b), and (c). In still other embodiments, the subject has an HbA_{1c} level of about 6% to about 10%, prior to receiving the combination of (a), (b), and (c). In some embodiments, the subject's HbA_{1c} level decreases by about 1% to about 5% after receiving the combination of (a), (b), and (c); for example, about 1% to about 2%, about 1.5% to about 2.5%, about 2% to about 3%, about 2.5% to about 3.5%, about 3% to about 4%, about 3.5% to about 4.5%, about 4% to about 5%, or about 1.5% to about 3%, or any value in between. In some embodiments, the subject's HbA_{1c} level decreases by about 1.5% to about 3% after receiving the combination of (a), (b), and (c). In some embodiments, the subject does not have Type I diabetes as a comorbidity. In other embodiments, the subject does not have Type II diabetes as a comorbidity.

In some embodiments, the subject has a mean fasting plasma glucose level of ≤170 mg/dL, ≤160 mg/dL, ≤150 mg/dL, ≤140 mg/dL, ≤130 mg/dL, ≤120 mg/dL, ≤110 mg/dL, or ≤100 mg/dL. In some embodiments, the subject has a mean fasting plasma glucose level, prior to receiving the combination of (a), (b), and (c), of about 90 mg/dL to about 110 mg/dL. In other embodiments, the subject has a mean fasting plasma glucose level, prior to receiving the combination of (a), (b), and (c), of about 100 mg/dL to about 120 mg/dL. In still other embodiments, the subject has a mean fasting plasma glucose level, prior to receiving the combination of (a), (b), and (c), of about 110 mg/dL to about 130 mg/dL. In some other embodiments, the subject has a mean fasting plasma glucose level, prior to receiving the combination of (a), (b), and (c), of about 120 mg/dL to about 140 mg/dL. In some embodiments, the subject has a mean fasting plasma glucose level, prior to receiving the combination of (a), (b), and (c), of about 130 mg/dL to about 150 mg/dL. In other embodiments, the subject has a mean fasting plasma glucose level, prior to receiving the combination of (a), (b), and (c), of about 140 mg/dL to about 160 mg/dL. In still other embodiments, the subject has a mean fasting plasma glucose level, prior to receiving the combination of (a), (b), and (c), of about 150 mg/dL to about 170 mg/dL. In some embodiments, the subject's mean fasting plasma glucose level decreases by about 30 mg/dL to about 90 mg/dL after receiving the combination of (a), (b), and (c); for example, by about 30 mg/dL to about 40 mg/dL, about 40 mg/dL to about 50 mg/dL, about 50 mg/dL to about 60 mg/dL, about 60 mg/dL to about 70 mg/dL, about 70 mg/dL to about 80 mg/dL, or about 80 mg/dL to about 90 mg/dL, or any value in between.

In some embodiments, the subject has a BMI of ≤35, ≤34, ≤33, ≤32, ≤31, ≤30, ≤29, ≤28, ≤27, ≤26, ≤25, ≤24, ≤23, ≤22, ≤21, or ≤20, or any value in between, prior to receiving the combination of (a), (b), and (c). In some embodiments, the subject has a BMI of about 35 to about 40, prior to receiving the combination of (a), (b), and (c). In other embodiments, the subject has a BMI of about 32 to about 35, prior to receiving the combination of (a), (b), and (c). In still other embodiments, the subject has a BMI of about 28 to about 32, prior to receiving the combination of (a), (b), and (c). In some other embodiments, the subject has a BMI of about 26 to about 30, prior to receiving the combination of (a), (b), and (c). In yet other embodiments, the subject has a BMI of about 24 to about 28, prior to receiving the combination of (a), (b), and (c). In some embodiments, the subject has a BMI of about 22 to about 26, prior to receiving the combination of (a), (b), and (c). In other embodiments, the subject has a BMI of about 20 to about 24, prior to receiving the combination of (a), (b), and (c). In some embodiments, the subject's BMI changes from about −10% to about +10% after receiving the combination of (a), (b), and (c). In some embodiments, the subject's BMI decreases by about 0% to about 10% after receiving the combination of (a), (b), and (c). In some embodiments, the subject's BMI decreases by about 0.5% to about 5% after receiving the combination of (a), (b), and (c). In some embodiments, the decrease in the subject's BMI occurs within about 4 weeks to about 104 weeks; for example, about 4 weeks to about 8 weeks, about 6 weeks to about 12 weeks, about 8 weeks to about 16 weeks, about 12 weeks to about 24 weeks, about 16 weeks to about 40 weeks, about 24 weeks to about 52 weeks, about 32 weeks to about 64 weeks, about 40 weeks to about 80 weeks, about 52 weeks to about 96 weeks, about 72 weeks to about 104 weeks, or any value in between.

In some embodiments, the subject's weight changes from about −10% to about +10% after receiving the combination of (a), (b), and (c). In some embodiments, the subject's weight changes from about −5% to about +5% after receiving the combination of (a), (b), and (c). In some embodiments, the subject's weight decreases by about 0% to about 10% after receiving the combination of (a), (b), and (c). In some embodiments, the subject's weight decreases by about 0.5% to about 5% after receiving the combination of (a), (b), and (c). In some embodiments, the subject's weight changes from about −5 kg to about +5 kg after receiving the combination of (a), (b), and (c). In some embodiments, the subject's weight changes from about −2 kg to about +2 kg after receiving the combination of (a), (b), and (c). In some embodiments, the subject's weight decreases by about 0 kg to about 5 kg after receiving the combination of (a), (b), and (c). In some embodiments, the subject's weight decreases by about 0.5 kg to about 2 kg after receiving the combination of (a), (b), and (c). In some embodiments, the changes in the subject's weight occurs within about 4 weeks to about 104 weeks; for example, about 4 weeks to about 8 weeks, about 6 weeks to about 12 weeks, about 8 weeks to about 16 weeks, about 12 weeks to about 24 weeks, about 16 weeks to about 40 weeks, about 24 weeks to about 52 weeks, about 32 weeks to about 64 weeks, about 40 weeks to about 80 weeks, about 52 weeks to about 96 weeks, about 72 weeks to about 104 weeks, or any value in between.

NAFLD is characterized by hepatic steatosis with no secondary causes of hepatic steatosis including excessive alcohol consumption, other known liver diseases, or long-term use of a steatogenic medication (Chalasani et al., Hepatology. 2018, 67(1):328-357, which is hereby incorporated by reference in its entirety). NAFLD can be categorized into non-alcoholic fatty liver (NAFL) and non-alcoholic steatohepatitis (NASH). According to Chalasani et al., NAFL is defined as the presence of ≥5% hepatic steatosis without evidence of hepatocellular injury in the form of hepatocyte ballooning. NASH is defined as the presence of ≥5% hepatic steatosis and inflammation with hepatocyte injury (e.g., ballooning), with or without any liver fibrosis. Additionally, NASH is commonly associated with hepatic inflammation and liver fibrosis, which can progress to cirrhosis, end-stage liver disease, and hepatocellular carcinoma. However, liver fibrosis is not always present in NASH, but the severity of fibrosis can be linked to long-term outcomes.

There are many approaches used to assess and evaluate whether a subject has NAFLD and if so, the severity of the disease including differentiating whether the NAFLD is NAFL or NASH.

For example, these approaches include determining one or more of hepatic steatosis (e.g., accumulation of fat in the liver); the NAFLD Activity Score (NAS); hepatic inflammation; biomarkers indicative of one or more of liver damage, hepatic inflammation, liver fibrosis, and/or liver cirrhosis (e.g., serum markers and panels); and liver fibrosis and/or cirrhosis. Further examples of physiological indicators of NAFLD can include liver morphology, liver stiffness, and the size or weight of the subject's liver. In some embodiments, NAFLD in the subject is evidenced by an accumulation of hepatic fat and detection of a biomarker indicative of liver damage. For example, elevated serum ferritin and low titers of serum autoantibodies can be common features of NAFLD. In some embodiments, methods to assess NAFLD include magnetic resonance imaging, either by spectroscopy or by proton density fat fraction (MRI-PDFF) to quantify steatosis, transient elastography (FIBROSCAN®), hepatic venous pressure gradient (HPVG), hepatic stiffness measurement with MRE for diagnosing significant liver fibrosis and/or cirrhosis, and assessing histological features of liver biopsy. In some embodiments, magnetic resonance imaging is used to detect one or more of steatohepatitis (NASH-MRI), liver fibrosis (Fibro-MRI), and steatosis see, e.g., U.S. Application Publication Nos. 2016/146715 and 2005/0215882, each of which are incorporated herein by reference in their entireties. In some embodiments, treatment of NAFLD comprises one or more of a decrease in symptoms; a reduction in the amount of hepatic steatosis; a decrease in the NAS; a decrease in hepatic inflammation; a decrease in the level of biomarkers indicative of one or more of liver damage, inflammation, liver fibrosis, and/or liver cirrhosis; and a reduction in fibrosis and/or cirrhosis, a lack of further progression of fibrosis and/or cirrhosis, or a slowing of the progression of fibrosis and/or cirrhosis.

In some embodiments, treatment of NAFLD comprises a decrease of one or more symptoms associated with NAFLD in the subject. Exemplary symptoms can include one or more of an enlarged liver, fatigue, pain in the upper right abdomen, abdominal swelling, enlarged blood vessels just beneath the skin's surface, enlarged breasts in men, enlarged spleen, red palms, jaundice, and pruritus. In some embodiments, the subject is asymptomatic. In some embodiments, the total body weight of the subject does not increase. In some embodiments, the total body weight of the subject decreases. In some embodiments, the body mass index (BMI) of the subject does not increase. In some embodiments, the body mass index (BMI) of the subject decreases. In some embodiments, the waist and hip (WTH) ratio of the subject does not increase. In some embodiments, the waist and hip (WTH) ratio of the subject decreases.

In some embodiments, hepatic steatosis is determined by one or more methods selected from the group consisting of ultrasonography, computed tomography (CT), magnetic resonance imaging, magnetic resonance spectroscopy (MRS), magnetic resonance elastography (MRE), transient elastography (TE) (e.g., FIBROSCAN®), measurement of liver size or weight, or by liver biopsy (see, e.g., Di Lascio et al., Ultrasound Med Biol. 2018 August; 44(8):1585-1596; Lv et al., J Clin Transl Hepatol. 2018 Jun. 28; 6(2): 217-221; Reeder, et al., J Magn Reson Imaging. 2011 October; 34(4): spcone; and de Lédinghen V, et al., J Gastroenterol Hepatol. 2016 April; 31(4):848-55, each of which are incorporated herein by reference in their entireties). A subject diagnosed with NAFLD can have more than about 5% hepatic steatosis, for example, about 5% to about 25%, about 25% to about 45%, about 45% to about 65%, or greater than about 65% hepatic steatosis. In some embodiments, a subject with about 5% to about 33% hepatic steatosis has stage 1 hepatic steatosis, a subject with about 33% to about 66% hepatic steatosis has stage 2 hepatic steatosis, and a subject with greater than about 66% hepatic steatosis has stage 3 hepatic steatosis. In some embodiments, treatment of NAFLD can be assessed by measuring hepatic steatosis. In some embodiments, treatment of NAFLD comprises a reduction in hepatic steatosis following administration of one or more compounds described herein.

In some embodiments, the amount of hepatic steatosis is determined prior to administration of the combination of (a) the compound of Formula (I), or a pharmaceutically acceptable salt thereof, (b) an SGLT-2 inhibitor, or a pharmaceutically acceptable salt or solvate thereof, and (c) a DPP-4 inhibitor, or a pharmaceutically acceptable salt thereof. In some embodiments, the amount of hepatic steatosis is determined during the period of time or after the period of time of administration of the combination of (a), (b), and (c). In some embodiments, a reduction in the amount of hepatic steatosis during the period of time or after the period of time of administration of the combination of (a), (b), and (c) compared to prior to administration of the combination of (a), (b), and (c) indicates treatment of NAFLD. For example, a reduction in the amount of hepatic steatosis by about 1% to about 50%, about 25% to about 75%, or about 50% to about 100% indicates treatment of NAFLD. In some embodiments, a reduction in the amount of hepatic steatosis by about 5%, bout 10%, about 15%, about 20%, about 25%, about 30%, about 35%, about 40%, about 45%, about 50%, about 55%, about 60%, about 65%, about 70%, about 75%, about 80%, about 85%, about 90%, or about 95% indicates treatment of NAFLD.

In some embodiments, the severity of NALFD can be assessed using the NAS. In some embodiments, treatment of NAFLD can be assessed using the NAS. In some embodiments, treatment of NAFLD comprises a reduction in the NAS following administration of one or more compounds described herein. In some embodiments, the NAS can be determined as described in Kleiner et al., Hepatology. 2005, 41(6):1313-1321, which is hereby incorporated by reference in its entirety. See, e.g., Table 2 for a simplified NAS scheme adapted from Kleiner.

TABLE 2
Example of the NAFLD Activity Score (NAS) with Fibrosis Stage

| Feature | Degree | Score |
|---|---|---|
| Steatosis | <5% | 0 |
| | 5-33% | 1 |
| | >33-66% | 2 |
| | >66% | 3 |
| Lobular | No foci | 0 |
| Inflammation | <2 foci/200x | 1 |
| | 2-4 foci/200x | 2 |
| | >4 foci/200x | 3 |
| Ballooning | None | 0 |
| degeneration | Few | 1 |
| | Many cells/Prominent ballooning | 2 |
| Fibrosis | None | 0 |
| | Perisinusoidal or periportal | 1 |
| | Perisinusoidal & portal/periportal | 2 |
| | Bridging fibrosis | 3 |
| | Cirrhosis | 4 |

In some embodiments, the NAS is determined non-invasively, for example, as described in U.S. Application Publication No. 2018/0140219, which is incorporated by reference herein in its entirety. In some embodiments, a NAS is determined for a sample from the subject prior to administration of the combination of (a) the compound of Formula (I), or a pharmaceutically acceptable salt thereof, (b) an SGLT-2 inhibitor, or a pharmaceutically acceptable salt or solvate thereof, and (c) a DPP-4 inhibitor, or a pharmaceutically acceptable salt thereof. In some embodiments, the NAS is determined during the period of time or after the period of time of administration of the combination of (a), (b), and (c). In some embodiments, a lower NAS score during the period of time or after the period of time of administration of the combination of (a), (b), and (c) compared to prior to administration of the combination of (a), (b), and (c) indicates treatment of NAFLD. For example, a decrease in the NAS by 1, by 2, by 3, by 4, by 5, by 6, or by 7 indicates treatment of NAFLD. In some embodiments, the NAS following administration of the combination of (a), (b), and (c) is 7 or less. In some embodiments, the NAS during the period of time of administration of the combination of (a), (b), and (c) is 5 or less, 4 or less, 3 or less, or 2 or less. In some embodiments, the NAS during the period of time of administration of the combination of (a), (b), and (c) is 7 or less. In some embodiments, the NAS during the period of time of administration of the combination of (a), (b), and (c) is 5 or less, 4 or less, 3 or less, or 2 or less. In some embodiments, the NAS after the period of time of administration of the combination of (a), (b), and (c) is 7 or less. In some embodiments, the NAS after the period of time of administration of the combination of (a), (b), and (c) is 5 or less, 4 or less, 3 or less, or 2 or less.

In some embodiments, the presence of hepatic inflammation is determined by one or more methods selected from the group consisting of biomarkers indicative of hepatic inflammation and a liver biopsy sample(s) from the subject. In some embodiments, the severity of hepatic inflammation is determined from a liver biopsy sample(s) from the subject. For example, hepatic inflammation in a liver biopsy sample can be assessed as described in Kleiner et al., Hepatology. 2005, 41(6):1313-1321 and Brunt et al., Am J Gastroenterol 1999, 94:2467-2474, each of which are hereby incorporated by reference in their entireties. In some embodiments, the severity of hepatic inflammation is determined prior to administration of the combination of (a) the compound of Formula (I), or a pharmaceutically acceptable salt thereof, (b) an SGLT-2 inhibitor, or a pharmaceutically acceptable salt or solvate thereof, and (c) a DPP-4 inhibitor, or a pharmaceutically acceptable salt thereof,. In some embodiments, the severity of hepatic inflammation is determined during the period of time or after the period of time of administration of the combination of (a), (b), and (c). In some embodiments, a decrease in the severity of hepatic inflammation during the period of time or after the period of time of administration of the combination of (a), (b), and (c) compared to prior to administration of the combination of (a), (b), and (c) indicates treatment of NAFLD. For example, a decrease in the severity of hepatic inflammation by about 1% to about 50%, about 25% to about 75%, or about 50% to about 100% indicates treatment of NAFLD. In some embodiments, a decrease in the severity of hepatic inflammation by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, or about 95% indicates treatment of NAFLD.

In some embodiments, treatment of NAFLD comprises treatment of fibrosis and/or cirrhosis, e.g., a decrease in the severity of fibrosis, a lack of further progression of fibrosis and/or cirrhosis, or a slowing of the progression of fibrosis and/or cirrhosis. In some embodiments, the presence of fibrosis and/or cirrhosis is determined by one or more methods selected from the group consisting of transient elastography (e.g., FIBROSCAN®), non-invasive markers of hepatic fibrosis, and histological features of a liver biopsy. In some embodiments, the severity (e.g., stage) of fibrosis is determined by one or more methods selected from the group consisting of transient elastography (e.g., FIBROSCAN®), a fibrosis-scoring system, biomarkers of hepatic fibrosis (e.g., non-invasive biomarkers), and hepatic venous pressure gradient (HVPG). Non-limiting examples of fibrosis scoring systems include the NAFLD fibrosis scoring system (see, e.g., Angulo, et al., Hepatology. 2007; 45(4):846-54), the fibrosis scoring system in Brunt et al., Am J Gastroenterol. 1999, 94:2467-2474, the fibrosis scoring system in Kleiner et al., Hepatology. 2005, 41(6):1313-1321, and the ISHAK fibrosis scoring system (see Ishak et al., J Hepatol. 1995; 22:696-9), the contents of each of which are incorporated by reference herein in their entireties.

In some embodiments, the severity of fibrosis is determined prior to administration of the combination of (a) the compound of Formula (I), or a pharmaceutically acceptable salt thereof, (b) an SGLT-2 inhibitor, or a pharmaceutically acceptable salt or solvate thereof, and (c) a DPP-4 inhibitor, or a pharmaceutically acceptable salt thereof. In some embodiments, the severity of fibrosis is determined during the period of time or after the period of time of administration of the combination of (a), (b), and (c). In some embodiments, a decrease in the severity of fibrosis during the period of time or after the period of time of administration of the combination of (a), (b), and (c) compared to prior to administration of the combination of (a), (b), and (c) indicates treatment of NAFLD. In some embodiments, a decrease in the severity of fibrosis, a lack of further progression of fibrosis and/or cirrhosis, or a slowing of the progression of fibrosis and/or cirrhosis indicates treatment of NAFLD. In some embodiments, the severity of fibrosis is determined using a scoring system such as any of the fibrosis scoring systems described herein, for example, the score can indicate the stage of fibrosis, e.g., stage 0 (no fibrosis), stage 1, stage 2, stage 3, and stage 4 (cirrhosis) (see, e.g., Kleiner et al). In some embodiments, a decrease in the stage of the fibrosis is a decrease in the severity of the fibrosis. For example, a decrease by 1, 2, 3, or 4 stages is a decrease in the severity of the fibrosis. In some embodiments, a decrease in the stage, e.g., from stage 4 to stage 3, from stage 4 to stage 2, from stage 4 to stage 1, from stage 4 to stage 0, from stage 3 to stage 2, from stage 3 to stage 1, from stage 3 to stage 0, from stage 2 to stage 1, from stage 2 to stage 0, or from stage 1 to stage 0 indicates treatment of NAFLD. In some embodiments, the stage of fibrosis decreases from stage 4 to stage 3, from stage 4 to stage 2, from stage 4 to stage 1, from stage 4 to stage 0, from stage 3 to stage 2, from stage 3 to stage 1, from stage 3 to stage 0, from stage 2 to stage 1, from stage 2 to stage 0, or from stage 1 to stage 0 following administration of the combination of (a), (b), and (c) compared to prior to administration of the combination of (a), (b), and (c). In some embodiments, the stage of fibrosis decreases from stage 4 to stage 3, from stage 4 to stage 2, from stage 4 to stage 1, from stage 4 to stage 0, from stage 3 to stage 2, from stage 3 to stage 1, from stage 3 to stage 0, from stage 2 to stage 1, from stage 2 to stage 0, or from stage 1 to stage 0 during the period of time of administration of the combination of (a), (b), and (c) compared to prior to administration of the combination of (a), (b), and (c). In some embodiments, the stage of fibrosis decreases from stage 4 to stage 3, from stage 4 to stage 2, from stage 4 to stage 1, from stage 4 to stage 0, from stage 3 to stage 2, from stage 3 to stage 1, from stage 3 to stage 0, from stage 2 to stage 1, from stage 2 to stage 0, or from stage 1 to stage 0 after the period of time of administration of the combination of (a), (b), and (c) compared to prior to administration of the combination of (a), (b), and (c).

In some embodiments, the presence of NAFLD is determined by one or more biomarkers indicative of one or more of liver damage, inflammation, liver fibrosis, and/or liver cirrhosis or scoring systems thereof. In some embodiments, the severity of NAFLD is determined by one or more biomarkers indicative of one or more of liver damage, inflammation, liver fibrosis, and/or liver cirrhosis or scoring systems thereof. The level of the biomarker can be determined by, for example, measuring, quantifying, and monitoring the expression level of the gene or mRNA encoding the biomarker and/or the peptide or protein of the biomarker. Non-limiting examples of biomarkers indicative of one or more of liver damage, inflammation, liver fibrosis, and/or liver cirrhosis and/or scoring systems thereof include the aspartate aminotransferase (AST) to platelet ratio index (APRI); the aspartate aminotransferase (AST) and alanine aminotransferase (ALT) ratio (AAR); the FIB-4 score, which is based on the APRI, alanine aminotransferase (ALT) levels, and age of the subject (see, e.g., McPherson et al., Gut. 2010 September; 59(9):1265-9, which is incorporated by reference herein in its entirety); hyaluronic acid; pro-inflammatory cytokines; a panel of biomarkers consisting of α2-macroglobulin, haptoglobin, apolipoprotein A1, bilirubin, gamma glutamyl transpeptidase (GGT) combined with a subject's age and gender to generate a measure of fibrosis and necroinflammatory activity in the liver (e.g., FIBROTEST®, FIBROSURE®), a panel of biomarkers consisting of bilirubin, gamma-glutamyltransferase, hyaluronic acid, α2-macroglobulin combined with the subject's age and sex (e.g., HEPASCORE®; see, e.g., Adams et al., Clin Chem. 2005 October; 51(10):1867-73), and a panel of biomarkers consisting of tissue inhibitor of metalloproteinase-1, hyaluronic acid, and α2-macroglobulin (e.g., FIBROSPECT®); a panel of biomarkers consisting of tissue inhibitor of metalloproteinases 1 (TIMP-1), amino-terminal propeptide of type III procollagen (PIIINP) and hyaluronic acid (HA) (e.g., the Enhanced Liver Fibrosis (ELF) score, see, e.g., Lichtinghagen R, et al., J Hepatol. 2013 August; 59(2):236-42, which is incorporated by reference herein in its entirety). In some embodiments, the presence of fibrosis is determined by one or more of the FIB-4 score, a panel of biomarkers consisting of α2-macroglobulin, haptoglobin, apolipoprotein A1, bilirubin, gamma glutamyl transpeptidase (GGT) combined with a subject's age and gender to generate a measure of fibrosis and necroinflammatory activity in the liver (e.g., FIBROTEST®, FIBROSURE®), a panel of biomarkers consisting of bilirubin, gamma-glutamyltransferase, hyaluronic acid, α2-macroglobulin combined with the subject's age and sex (e.g., HEPASCORE®; see, e.g., Adams et al., Clin Chem. 2005 October; 51(10):1867-73), and a panel of biomarkers consisting of tissue inhibitor of metalloproteinase-1, hyaluronic acid, and α2-macroglobulin (e.g., FIBROSPECT®); and a panel of biomarkers consisting of tissue inhibitor of metalloproteinases 1 (TIMP-1), amino-terminal propeptide of type III procollagen (PIIINP) and hyaluronic acid (HA) (e.g., the Enhanced Liver Fibrosis (ELF) score).

In some embodiments, the level of aspartate aminotransferase (AST) does not increase. In some embodiments, the level of aspartate aminotransferase (AST) decreases. In some embodiments, the level of alanine aminotransferase (ALT) does not increase. In some embodiments, the level of alanine aminotransferase (ALT) decreases. In some embodiments, the “level” of an enzyme refers to the concentration of the enzyme, e.g., within blood. For example, the level of AST or ALT can be expressed as Units/L.

In some embodiments, the severity of fibrosis is determined by one or more of the FIB-4 score, a panel of biomarkers consisting of α2-macroglobulin, haptoglobin, apolipoprotein A1, bilirubin, gamma glutamyl transpeptidase (GGT) combined with a subject's age and gender to generate a measure of fibrosis and necroinflammatory activity in the liver (e.g., FIBROTEST®, FIBROSURE®), a panel of biomarkers consisting of bilirubin, gamma-glutamyltransferase, hyaluronic acid, α2-macroglobulin combined with the subject's age and sex (e.g., HEPASCORE®; see, e.g., Adams et al.,Clin Chem. 2005 October; 51(10):1867-73, which is incorporated by reference herein in its entirety), and a panel of biomarkers consisting of tissue inhibitor of metalloproteinase-1, hyaluronic acid, and α2-macroglobulin (e.g., FIBROSPECT®); and a panel of biomarkers consisting of tissue inhibitor of metalloproteinases 1 (TIMP-1), amino-terminal propeptide of type III procollagen (PIIINP) and hyaluronic acid (HA) (e.g., the Enhanced Liver Fibrosis (ELF) score).

In some embodiments, hepatic inflammation is determined by the level of liver inflammation biomarkers, e.g., pro-inflammatory cytokines. Non-limiting examples of biomarkers indicative of liver inflammation include interleukin-(IL) 6, interleukin-(IL) 1β, tumor necrosis factor (TNF)-α, transforming growth factor (TGF)-β, monocyte chemotactic protein (MCP)-1, C-reactive protein (CRP), PAI-1, and collagen isoforms such as Col1a1, Col1a2, and Col4a1 (see, e.g., Neuman, et al., Can J Gastroenterol Hepatol. 2014 December; 28(11): 607-618 and U.S. Pat. No. 9,872,844, each of which are incorporated by reference herein in their entireties). Liver inflammation can also be assessed by change of macrophage infiltration, e.g., measuring a change of CD68 expression level. In some embodiments, liver inflammation can be determined by measuring or monitoring serum levels or circulating levels of one or more of interleukin-(IL) 6, interleukin-(IL) 1β, tumor necrosis factor (TNF)-α, transforming growth factor (TGF)-β, monocyte chemotactic protein (MCP)-1, and C-reactive protein (CRP).

In some embodiments, the level of one or more biomarkers indicative of one or more of liver damage, inflammation, liver fibrosis, and/or liver cirrhosis is determined for a sample from the subject prior to administration of the combination of (a) the compound of Formula (I), or a pharmaceutically acceptable salt thereof, (b) an SGLT-2 inhibitor, or a pharmaceutically acceptable salt or solvate thereof, and (c) a DPP-4 inhibitor, or a pharmaceutically acceptable salt thereof. In some embodiments, the level of one or more biomarkers indicative of one or more of liver damage, inflammation, liver fibrosis, and/or liver cirrhosis is determined during the period of time or after the period of time of administration of the combination of (a), (b), and (c). In some embodiments, a decrease in the level of one or more biomarkers indicative of one or more of liver damage, inflammation, liver fibrosis, and/or liver cirrhosis during the period of time or after the period of time of administration of the combination of (a), (b), and (c) compared to prior to administration of the combination of (a), (b), and (c) indicates treatment of NAFLD. For example, a decrease in the level of one or more biomarkers indicative of one or more of liver damage, inflammation, liver fibrosis, and/or liver cirrhosis by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 99% indicates treatment of NAFLD. In some embodiments, the decrease in the level of one or more biomarkers indicative of one or more of liver damage, inflammation, liver fibrosis, and/or liver cirrhosis following administration of the combination of (a), (b), and (c) is by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 99%. In some embodiments, the level of one or more biomarkers indicative of one or more of liver damage, inflammation, liver fibrosis, and/or liver cirrhosis during the period of time of administration of the combination of (a), (b), and (c) is by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 99%. In some embodiments, the level of one or more biomarkers indicative of one or more of liver damage, inflammation, liver fibrosis, and/or liver cirrhosis after the period of time of administration of the combination of (a), (b), and (c) is by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 99%.

In some embodiments, the treatment of NAFLD decreases the level of serum bile acids in the subject. In some embodiments, the level of serum bile acids is determined by, for example, an ELISA enzymatic assay or the assays for the measurement of total bile acids as described in Danese et al., PLoS One. 2017; 12(6): e0179200, which is incorporated by reference herein in its entirety. In some embodiments, the level of serum bile acids can decrease by, for example, 10% to 40%, 20° A to 50%, 30° A to 60%, 40° A to 70%, 50% to 80%, or by more than 90% of the level of serum bile acids prior to administration of (a), (b), and (c). In some embodiments, the NAFLD is NAFLD with attendant cholestasis. In cholestasis, the release of bile, including bile acids, from the liver is blocked. Bile acids can cause hepatocyte damage (see, e.g., Perez M J, Briz O. World J Gastroenterol. 2009 Apr. 14; 15(14):1677-89) likely leading to or increasing the progression of fibrosis (e.g., cirrhosis) and increasing the risk of hepatocellular carcinoma (see, e.g., Sorrentino P et al. Dig Dis Sci. 2005 June; 50(6):1130-5 and Satapathy S K and Sanyal A J. Semin Liver Dis. 2015, 35(3):221-35, each of which are incorporated by reference herein in their entireties). In some embodiments, the NAFLD with attendant cholestasis is NASH with attendant cholestasis. In some embodiments, the treatment of NAFLD comprises treatment of pruritus. In some embodiments, the treatment of NAFLD with attendant cholestasis comprises treatment of pruritus. In some embodiments, a subject with NAFLD with attendant cholestasis has pruritus.

In some embodiments, treatment of NAFLD comprises an increase in adiponectin. It is thought that the compound of Formula (I) may be a selective activator of a highly limited number of PPARγ pathways including pathways regulated by adiponectin. Adiponectin is an anti-fibrotic and anti-inflammatory adipokine in the liver (see e.g., Park et al., Curr Pathobiol Rep. 2015 Dec. 1; 3(4): 243-252.). In some embodiments, the level of adiponectin is determined by, for example, an ELISA enzymatic assay. In some embodiments, the adiponectin level in the subject is increased by at least about 30%, at least about 68%, at least about 175%, or at least about 200%. In some embodiments, the increase is by at least about 175%. In some embodiments, the level of adiponectin is determined for a sample from the subject prior to administration of the combination of (a) the compound of Formula (I), or a pharmaceutically acceptable salt thereof, (b) an SGLT-2 inhibitor, or a pharmaceutically acceptable salt or solvate thereof, and (c) a DPP-4 inhibitor, or a pharmaceutically acceptable salt thereof. In some embodiments, the level of adiponectin is determined during the period of time or after the period of time of administration of the combination of (a), (b), and (c). In some embodiments, an increase in the level of adiponectin during the period of time or after the period of time of administration of the combination of (a), (b), and (c) compared to prior to administration of the combination of (a), (b), and (c) indicates treatment of NAFLD. For example, an increase in the level of adiponectin by at least about 30%, at least about 68%, at least about 175%, or at least about 200% indicates treatment of NAFLD. In some embodiments, the increase in the level of adiponectin following administration of the combination of (a), (b), and (c) is at least about 200%.

Provided herein are methods of treating non-alcoholic fatty liver disease (NAFLD) in a subject in need thereof comprising or consisting essentially of administering to the subject (a) the compound of Formula (I), or a pharmaceutically acceptable salt thereof, (b) an SGLT-2 inhibitor, or a pharmaceutically acceptable salt or solvate thereof, and (c) a DPP-4 inhibitor, or a pharmaceutically acceptable salt thereof, wherein the amounts of (a), (b), and (c) together are effective in treating NAFLD. In some embodiments, a method of treating non-alcoholic fatty liver disease (NAFLD) in a subject in need thereof comprises or consists essentially of administering to the subject (a) the compound of Formula (I), or a pharmaceutically acceptable salt thereof, (b) an SGLT-2 inhibitor, or a pharmaceutically acceptable salt or solvate thereof, and (c) a DPP-4 inhibitor, or a pharmaceutically acceptable salt thereof, during a period of time, wherein the amounts of (a), (b), and (c) together are effective in treating NAFLD.

Also provided herein are methods of treating a subject, the method comprising: selecting a subject having non-alcoholic fatty liver disease (NAFLD); and administering (a) the compound of Formula (I), or a pharmaceutically acceptable salt thereof, (b) an SGLT-2 inhibitor, or a pharmaceutically acceptable salt or solvate thereof, and (c) a DPP-4 inhibitor, or a pharmaceutically acceptable salt thereof, to the selected subject, wherein the amounts of (a), (b), and (c) together are effective in treating NAFLD. In some embodiments, (a), (b), and (c) are administered during a period of time.

Also provided herein are methods of treating a subject, the method comprising: identifying a subject having non-alcoholic fatty liver disease (NAFLD); and administering (a) the compound of Formula (I), or a pharmaceutically acceptable salt thereof, (b) an SGLT-2 inhibitor, or a pharmaceutically acceptable salt or solvate thereof, and (c) a DPP-4 inhibitor, or a pharmaceutically acceptable salt thereof, to the selected subject, wherein the amounts of (a), (b), and (c) together are effective in treating NAFLD. In some embodiments, (a), (b), and (c) are administered during a period of time.

Provided herein are methods of treating NAFLD in a subject in need thereof comprising or consisting essentially of administering to the subject (a) a therapeutically effective amount of the compound of Formula (I), or a pharmaceutically acceptable salt thereof, (b) a therapeutically effective amount of an SGLT-2 inhibitor, or a pharmaceutically acceptable salt or solvate thereof, and (c) a therapeutically effective amount of a DPP-4 inhibitor, or a pharmaceutically acceptable salt thereof In some embodiments, a method of treating NAFLD in a subject in need thereof comprises or consists essentially of administering to the subject (a) the compound of Formula (I), or a pharmaceutically acceptable salt thereof, (b) an SGLT-2 inhibitor, or a pharmaceutically acceptable salt or solvate thereof, and (c) a DPP-4 inhibitor, or a pharmaceutically acceptable salt thereof, during a period of time. In some embodiments, the amounts of (a), (b), and (c) together are effective in treating NAFLD.

Also provided herein are methods of treating a subject, the method comprising: selecting a subject having non-alcoholic fatty liver disease (NAFLD); and administering (a) the compound of Formula (I), or a pharmaceutically acceptable salt thereof, (b) an SGLT-2 inhibitor, or a pharmaceutically acceptable salt or solvate thereof, and (c) a DPP-4 inhibitor, or a pharmaceutically acceptable salt thereof, to the selected subject. In some embodiments, (a), (b), and (c) are administered during a period of time. In some embodiments, the amounts of (a), (b), and (c) together are effective in treating NAFLD.

Also provided here are methods of selecting a subject for treatment, the method comprising: identifying a subject having NAFLD; and selecting the identified subject for treatment with (a) the compound of Formula (I), or a pharmaceutically acceptable salt thereof, (b) an SGLT-2 inhibitor, or a pharmaceutically acceptable salt or solvate thereof, and (c) a DPP-4 inhibitor, or a pharmaceutically acceptable salt thereof. In some embodiments, the amounts of (a), (b), and (c) together are effective in treating NAFLD.

Also provided herein are methods of selecting a subject for participation in a clinical trial, the method comprising: identifying a subject having NAFLD; and selecting the identified subject for participation in a clinical trial that comprises administration of (a) the compound of Formula (I), or a pharmaceutically acceptable salt thereof, (b) an SGLT-2 inhibitor, or a pharmaceutically acceptable salt or solvate thereof, and (c) a DPP-4 inhibitor, or a pharmaceutically acceptable salt thereof. In some embodiments, the amounts of (a), (b), and (c) together are effective in treating NAFLD.

In some embodiments, (a), (b), and (c) are administered concurrently. In some embodiments, (a), (b), and (c) are administered as a fixed combination. In some embodiments(a), (b), and (c) are administered as a non-fixed combination. In some embodiments, (a), (b), and (c) are administered sequentially and in any order, at specific or varying time intervals (e.g., during the period of time). In some embodiments, a therapeutically effective amount of each of (a), (b), and (c) are administered concurrently. In some embodiments, a therapeutically effective amount of each of (a), (b), and (c) are administered sequentially and in any order, at specific or varying time intervals (e.g., during the period of time).

In some embodiments, the amount of the compound of Formula (I), or a pharmaceutically acceptable salt thereof, is from about 0.1 to about 15 milligrams (mg). For example, from about 0.1 to about 10 mg, about 5 to about 15 mg, or about 2 to about 12 mg. In some embodiments, the compound of Formula (I), or a pharmaceutically acceptable salt thereof, is administered at a dose from about 0.1 to about 5 mg, about 0.1 to about 4 mg, about 0.5 to about 3 mg, about 0.5 to about 2 mg, about 0.5 to about 1 mg, about 1 to about 3 mg, about 2 to about 4 mg, about 3 to about 5 mg, about 1 to about 6 mg, about 2 to about 6 mg, about 3 to about 6 mg, about 4 to about 6 mg, or about 5 to about 6 mg. In some embodiments, the dose is a therapeutically effective amount.

In some embodiments, the compound of Formula (I), or a pharmaceutically acceptable salt thereof, is administered to the subject twice a day, daily, every other day, three times a week, twice a week, weekly, every other week, twice a month, or monthly. In some embodiments, the compound of Formula (I), or a pharmaceutically acceptable salt thereof, is administered to the subject daily.

In some embodiments, the compound of Formula (I), or a pharmaceutically acceptable salt thereof, is administered to the subject daily and at a dose of about 3 mg. In some embodiments, the compound of Formula (I), or a pharmaceutically acceptable salt thereof, is administered at a dose from about 0.1 to about 10.0 mg per day. In some embodiments, the compound of Formula (I), or a pharmaceutically acceptable salt thereof, is administered at a dose from about 0.1 to about 3 mg per day. In some embodiments, the compound of Formula (I), or a pharmaceutically acceptable salt thereof, is administered at a dose about 0.5 milligram per day. In some embodiments, the compound of Formula (I), or a pharmaceutically acceptable salt thereof, is administered at a dose about 1 milligram per day. In some embodiments, the compound of Formula (I), or a pharmaceutically acceptable salt thereof, is administered at a dose about 2 mg per day.

In some embodiments, the compound of Formula (I) is in the form of a besylate salt. In some embodiments, the compound of Formula (I) is in the form of an HCl salt. In some embodiments, the compound of Formula (I) is in the form of an HBr salt. In some embodiments, the compound of Formula (I) is in the form of a tosylate salt.

In some embodiments, the amount of the SGLT-2 inhibitor, or a pharmaceutically acceptable salt or solvate thereof, is from about 1 to about 350 mg. For example, about 1 to about 175 mg, about 175 to about 350 mg, or about 90 to about 260 mg. In some embodiments, the amount of the SGLT-2 inhibitor, or a pharmaceutically acceptable salt or solvate thereof, is from about 85 to about 325 mg. In some embodiments, the amount of the SGLT-2 inhibitor, or a pharmaceutically acceptable salt or solvate thereof, is from about 1 to about 50 mg, about 20 to about 70 mg, about 50 to about 100 mg, about 70 to about 120 mg, about 90 to about 140 mg, about 110 to about 160 mg, about 130 to about 180 mg, about 150 to about 200 mg, about 170 to about 220 mg, about 190 to about 240 mg, about 210 to about 260 mg, about 230 to about 280 mg, about 250 to about 300 mg, about 270 to about 320 mg, or about 290 to about 350 mg. For example, about 100 mg or about 300 mg.

In some embodiments, the SGLT-2 inhibitor is selected from the group consisting of: empagliflozin, canagliflozin, dapagliflozin, ertugliflozin, ipragliflozin, luseogliflozin, remogliflozin etabonate, serfliflozin etabonate, sotagliflozin, tofogliflozin, or a combination of two or more thereof. In some embodiments, the SGLT-2 inhibitor is empagliflozin.

In some embodiments, the SGLT-2 inhibitor, or a pharmaceutically acceptable salt or solvate thereof, is administered to the subject twice a day, daily, every other day, three times a week, twice a week, weekly, every other week, twice a month, or monthly. In some embodiments, the SGLT-2 inhibitor, or a pharmaceutically acceptable salt or solvate thereof, is administered to the subject daily.

In some embodiments, the SGLT-2 inhibitor is canagliflozin. In some embodiments, 100 mg or 300 mg of canagliflozin is administered. In some embodiments, the SGLT-2 inhibitor is dapagliflozin. In some embodiments, 5 mg or 10 mg of dapagliflozin is administered. In some embodiments, the SGLT-2 inhibitor is empagliflozin. In some embodiments, 10 mg or 25 mg of empagliflozin is administered. In some embodiments, the SGLT-2 inhibitor is ertugliflozin. In some embodiments, 5 mg or 15 mg of ertugliflozin is administered. In some embodiments, the SGLT-2 inhibitor is ipragliflozin. In some embodiments, 25 mg or 50 mg of ipragliflozin is administered. In some embodiments, the SGLT-2 inhibitor is bexagliflozin. In some embodiments, 20 mg of bexagliflozin is administered. In some embodiments, the SGLT-2 inhibitor is sotagliflozin. In some embodiments, 200 mg or 400 mg of sotagliflozin is administered. In some embodiments, the SGLT-2 inhibitor is licogliflozin. In some embodiments, 15 mg, 50 mg, 75 mg or 150 mg of licogliflozin is administered.

In some embodiments, the amount of the DPP-4 inhibitor, or a pharmaceutically acceptable salt thereof, is from about 1 to about 400 mg. For example, about 1 mg to about 20 mg, about 5 mg to about 50 mg, about 25 mg to about 100 mg, about 50 mg to about 200 mg, or about 100 mg to about 400 mg.

In some embodiments, the DPP-4 inhibitor is selected from the group consisting of: sitagliptin, vildagliptin, saxagliptin, linagliptin, gemigliptin, anagliptin, teneligliptin, alogliptin, trelagliptin, omarigliptin, evogliptin, and dutogliptin, or pharmaceutically acceptable salt of any of the foregoing. In some embodiments, the DPP-4 inhibitor is sitagliptin.

In some embodiments, the DPP-4 inhibitor, or a pharmaceutically acceptable salt thereof, is administered to the subject twice a day, daily, every other day, three times a week, twice a week, weekly, every other week, twice a month, or monthly. In some embodiments, the DPP-4 inhibitor, or a pharmaceutically acceptable salt thereof, is administered to the subject daily. In other embodiments, the DPP-4 inhibitor, or a pharmaceutically acceptable salt thereof, is administered to the subject weekly.

In some embodiments, the DPP-4 inhibitor is sitagliptin. In some embodiments, 25 mg, 50 mg, 75 mg, or 100 mg of sitagliptin is administered. In some embodiments, the DPP-4 inhibitor is vildagliptin. In some embodiments, 25 mg, 50 mg, 75 mg, or 100 mg of vildagliptin is administered. In some embodiments, the DPP-4 inhibitor is saxagliptin. In some embodiments, 1 mg, 2 mg, 2.5 mg, or 5 mg of saxagliptin is administered. In some embodiments, the DPP-4 inhibitor is linagliptin. In some embodiments, 1 mg, 2 mg, 2.5 mg, or 5 mg of linagliptin is administered. In some embodiments, the DPP-4 inhibitor is gemigliptin. In some embodiments, 10 mg, 25 mg, or 50 mg of gemigliptin is administered. In some embodiments, the DPP-4 inhibitor is anagliptin. In some embodiments, 50 mg, 100 mg, 150 mg, or 200 mg of anagliptin is administered. In some embodiments, the DPP-4 inhibitor is teneligliptin. In some embodiments, 5 mg, 10 mg, 20 mg, or 40 mg of teneligliptin is administered. In some embodiments, the DPP-4 inhibitor is alogliptin. In some embodiments, 5 mg, 10 mg, 15 mg, or 25 mg of alogliptin is administered. In some embodiments, the DPP-4 inhibitor is trelagliptin. In some embodiments, 10 mg, 25 mg, 50 mg, or 100 mg of trelagliptin is administered. In some embodiments, the DPP-4 inhibitor is omarigliptin. In some embodiments, 5 mg, 10 mg, 15 mg, or 25 mg of omarigliptin is administered. In some embodiments, the DPP-4 inhibitor is evogliptin. In some embodiments, 1 mg, 2.5 mg, 5 mg, or 10 mg of evogliptin is administered. In some embodiments, the DPP-4 inhibitor is dutogliptin. In some embodiments, 50 mg, 100 mg, 200 mg, or 400 mg of dutogliptin is administered.

In some embodiments, treatment of NAFLD comprises a decrease of one or more symptoms associated with NAFLD in the subject. Exemplary symptoms can include one or more of an enlarged liver, fatigue, pain in the upper right abdomen, abdominal swelling, enlarged blood vessels just beneath the skin's surface, enlarged breasts in men, enlarged spleen, red palms, jaundice, and pruritus. In some embodiments, the subject is asymptomatic.

In some embodiments, the treatment of NAFLD, e.g., NAFL or NASH, comprises a reduction in hepatic steatosis. For example, hepatic steatosis is decreased by at least 2%, 3%, 4%, 5%, 6%, 7%, 8%. 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, 96%, 97%, 98%, 99%, or more than 99% following administration of (a), (b), and (c) for a period of time.

In some embodiments, the treatment of NAFLD, e.g., NAFL or NASH, is assessed using the NAFLD Activity Score (NAS). In some embodiments, treatment of NAFLD comprises a decrease in the NAS. In some embodiments, the NAS for a sample from the subject following administration is 7 or less. In some embodiments, the NAS for a sample from the subject following administration is 5 or less, 4 or less, 3 or less, or 2 or less. In some embodiments, the NAFLD activity score (NAS) for a sample from the subject following administration during the period of time is 7 or less. In some embodiments, the NAS for a sample from the subject following administration during the period of time is 5 or less, 4 or less, 3 or less, or 2 or less. In some embodiments, the sample from the subject is from a liver biopsy.

In some embodiments, the treatment of NAFLD, e.g., NAFL or NASH, can be assessed using the NAFLD Activity Score (NAS). In some embodiments, the NAS for a sample from the subject following administration is reduced by 1 or more, 2 or more, 3 or more, 4 or more, 5 or more, or 6 or more. In some embodiments, the NAS for a sample from the subject following administration is reduced by 1, 2, 3, 4, 5, or 6. In some embodiments, the NAFLD activity score (NAS) for a sample from the subject following administration during the period of time is reduced by 1 or more, 2 or more, 3 or more, 4 or more, 5 or more, or 6 or more. In some embodiments, the NAS for a sample from the subject following administration during the period of time is reduced by 1, 2, 3, 4, 5, or 6. In some embodiments, the sample from the subject is from a liver biopsy.

In some embodiments, the treatment of NAFLD, e.g., NAFL or NASH, comprises treatment of hepatic inflammation. In some embodiments, the severity of the hepatic inflammation is decreased by about 1% to about 50%, about 25% to about 75%, or about 50% to about 100%. In some embodiments, the severity of hepatic inflammation is decreased by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, or about 95%.

In some embodiments, the treatment of NAFLD, e.g., NAFL or NASH, comprises treatment of fibrosis. In some embodiments, the treatment of the NAFLD comprises treatment of cirrhosis (e.g., stage 4 of fibrosis). In some embodiments, treatment of fibrosis comprises a decrease in the stage of fibrosis, for example, from stage 4 to stage 3, from stage 4 to stage 2, from stage 4 to stage 1, from stage 4 to stage 0, from stage 3 to stage 2, from stage 3 to stage 1, from stage 3 to stage 0, from stage 2 to stage 1, from stage 2 to stage 0, or from stage 1 to stage 0.

In some embodiments, the adiponectin level in the subject is increased by at least about 30%, at least about 68%, at least about 175%, or at least about 200%. In some embodiments, the increase is by at least about 175%.

In some embodiments, the level of aspartate aminotransferase (AST) in the subject does not increase. In some embodiments, the level of aspartate aminotransferase (AST) in the subject decreases. In some embodiments, the level of alanine aminotransferase (ALT) in the subject does not increase. In some embodiments, the level of alanine aminotransferase (ALT) in the subject decreases. In some embodiments, the total body weight of the subject does not increase. In some embodiments, the total body weight of the subject decreases. In some embodiments, the body mass index (BMI) of the subject does not increase. In some embodiments, the body mass index (BMI) of the subject decreases. In some embodiments, the waist and hip (WTH) ratio of the subject does not increase. In some embodiments, the waist and hip (WTH) ratio of the subject decreases.

In some embodiments, a non-invasive liver fibrosis marker does not increase or decreases. In some embodiments, the non-invasive liver fibrosis marker is Enhanced Liver Fibrosis (ELF) panel.

In some embodiments, treatment of NAFLD comprises a decrease in the level of one or more biomarkers indicative of one or more of liver damage, inflammation, fibrosis, and/or cirrhosis, e.g., any of the biomarkers as described herein. In some embodiments, treatment of NAFLD comprises a decrease in the level of one or more biomarkers indicative of one or more of liver damage, inflammation, fibrosis, and/or cirrhosis by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 99%.

In some embodiments, the treatment of NAFLD decreases the level of serum bile acids in the subject. In some embodiments, the treatment of NAFLD comprises treatment of pruritus.

In some embodiments, the subject has liver fibrosis associated with the NAFLD. In some embodiments, the subject has hepatic cirrhosis (e.g., stage 4 fibrosis) associated with the NAFLD. In some embodiments, the subject has liver fibrosis as a comorbidity. In some embodiments, the subject has hepatic cirrhosis (e.g., stage 4 fibrosis) as a comorbidity. In some embodiments, the subject has liver fibrosis caused by the NAFLD. In some embodiments, the subject has hepatic cirrhosis (e.g., stage 4 fibrosis) caused by the NAFLD.

In some embodiments, the NAFLD is simple nonalcoholic fatty liver (NAFL). In some embodiments, the NAFLD is NAFL with attendant liver fibrosis. In some embodiments, the NAFLD is NAFL with attendant liver cirrhosis.

In some embodiments, the NAFLD is nonalcoholic steatohepatitis (NASH). In some embodiments, the NAFLD is NASH with attendant liver fibrosis. In some embodiments, the NAFLD is NASH with attendant liver cirrhosis.

In some embodiments, the method further comprises performing a liver biopsy to determine the NAFLD activity score of the biopsy sample obtained from the subject.

In some embodiments, (a), (b), and (c) are administered prophylactically.

In some embodiments, the subject was previously treated, before the period of time, with one or more therapeutic agents, e.g., treatment with at least one NAFLD treatment, NASH treatment, type 2 diabetes treatment, obesity treatment, metabolic syndrome treatment, liver disease treatment, cardiovascular treatment, heart failure treatment, hypertension treatment. In some embodiments, the one or more therapeutic agents that were administered to the subject before the period of time was unsuccessful (e.g., therapeutically unsuccessful as determined by a physician). In some embodiments, the unsuccessful treatment did not comprises or consist essentially of administration of (a), (b), and (c).

In some embodiments, the method of treating non-alcoholic fatty liver disease (NAFLD) in a subject in need thereof comprises or consists essentially of administering to the subject (a) the compound of Formula (I), or a pharmaceutically acceptable salt thereof, (b) empagliflozin, or a pharmaceutically acceptable salt thereof, and (c) sitagliptin, wherein the amounts of (a), (b), and (c) together are effective in treating NAFLD. In some embodiments, a method of treating non-alcoholic fatty liver disease (NAFLD) in a subject in need thereof comprises or consists essentially of administering to the subject (a) the compound of Formula (I), or a pharmaceutically acceptable salt thereof, and (b) empagliflozin, or a pharmaceutically acceptable salt thereof, during a period of time, wherein the amounts of (a), (b), and (c) together are effective in treating NAFLD.

In some embodiments, the method of treating NAFLD in a subject in need thereof comprises or consists essentially of administering to the subject a therapeutically effective amount of (a) the compound of Formula (I), or a pharmaceutically acceptable salt thereof, and (b) empagliflozin, or a pharmaceutically acceptable salt thereof, and (c) sitagliptin. In some embodiments, a method of treating NAFLD in a subject in need thereof comprises or consists essentially of administering to the subject a therapeutically effective amount of (a) the compound of Formula (I), or a pharmaceutically acceptable salt thereof, (b) empagliflozin, or a pharmaceutically acceptable salt thereof, and (c) sitagliptin, during a period of time.

Also provided herein are methods of treating fibrosis in a subject in need thereof comprising or consisting essentially of administering to the subject (a) the compound of Formula (I), or a pharmaceutically acceptable salt thereof, (b) an SGLT-2 inhibitor, or a pharmaceutically acceptable salt or solvate thereof, and (c) sitagliptin, wherein the amounts of (a), (b), and (c) together are effective in treating fibrosis. In some embodiments, a method of treating fibrosis in a subject in need thereof comprises or consists essentially of administering to the subject (a) the compound of Formula (I), or a pharmaceutically acceptable salt thereof, (b) an SGLT-2 inhibitor, or a pharmaceutically acceptable salt or solvate thereof, and (c) sitagliptin, during a period of time, wherein the amounts of (a), (b), and (c) together are effective in treating fibrosis.

Provided herein are methods of treating fibrosis in a subject in need thereof comprising or consisting essentially of administering to the subject (a) a therapeutically effective amount of the compound of Formula (I), or a pharmaceutically acceptable salt thereof, and (b) a therapeutically effective amount of an SGLT-2 inhibitor, or a pharmaceutically acceptable salt or solvate thereof, and (c) sitagliptin. In some embodiments, a method of treating fibrosis in a subject in need thereof comprises or consists essentially of administering to the subject (a) a therapeutically effective amount of the compound of Formula (I), or a pharmaceutically acceptable salt thereof, and (b) a therapeutically effective amount of an SGLT-2 inhibitor, or a pharmaceutically acceptable salt or solvate thereof, and (c) a DPP-4 inhibitor, or a pharmaceutically acceptable salt thereof, during a period of time. In some embodiments, the amounts of (a), (b), and (c) together are effective in treating fibrosis.

In some embodiments, the fibrosis is cirrhosis (e.g., stage 4 of fibrosis). In some embodiments, the fibrosis is associated with NAFLD (e.g., NAFL or NASH). In some embodiments, the cirrhosis is associated with the NAFLD (e.g., NAFL or NASH). In some embodiments, the fibrosis is caused by NAFLD (e.g., NAFL or NASH). In some embodiments, the cirrhosis is caused by the NAFLD (e.g., NAFL or NASH).

In some embodiments, the treatment of fibrosis comprises a decrease in the severity of the fibrosis, a lack of progression of the fibrosis, or a slowing of the progression of the fibrosis. In some embodiments, treatment of fibrosis comprises a decrease in the stage of fibrosis, for example, from stage 4 to stage 3, from stage 4 to stage 2, from stage 4 to stage 1, from stage 4 to stage 0, from stage 3 to stage 2, from stage 3 to stage 1, from stage 3 to stage 0, from stage 2 to stage 1, from stage 2 to stage 0, or from stage 1 to stage 0.

Also provided herein are methods of treating hepatic steatosis in a subject in need thereof comprising or consisting essentially of administering to the subject (a) the compound of Formula (I), or a pharmaceutically acceptable salt thereof, (b) an SGLT-2 inhibitor, or a pharmaceutically acceptable salt or solvate thereof, and (c) a DPP-4 inhibitor, or a pharmaceutically acceptable salt thereof, wherein the amounts of (a), (b), and (c) together are effective in treating hepatic steatosis. In some embodiments, a method of treating hepatic steatosis in a subject in need thereof comprises or consists essentially of administering to the subject (a) the compound of Formula (I), or a pharmaceutically acceptable salt thereof, (b) an SGLT-2 inhibitor, or a pharmaceutically acceptable salt or solvate thereof, and (c) a DPP-4 inhibitor, or a pharmaceutically acceptable salt thereof, during a period of time, wherein the amounts of (a), (b), and (c) together are effective in treating hepatic steatosis.

Provided herein are methods of treating hepatic steatosis in a subject in need thereof comprising or consisting essentially of administering to the subject (a) a therapeutically effective amount of the compound of Formula (I), or a pharmaceutically acceptable salt thereof, (b) an SGLT-2 inhibitor, or a pharmaceutically acceptable salt or solvate thereof, and (c) a DPP-4 inhibitor, or a pharmaceutically acceptable salt thereof. In some embodiments, a method of treating hepatic steatosis in a subject in need thereof comprises or consists essentially of administering to the subject (a) a therapeutically effective amount of the compound of Formula (I), or a pharmaceutically acceptable salt thereof, (b) an SGLT-2 inhibitor, or a pharmaceutically acceptable salt or solvate thereof, and (c) a DPP-4 inhibitor, or a pharmaceutically acceptable salt thereof, during a period of time. In some embodiments, the amounts of (a), (b), and (c) together are effective in treating hepatic steatosis.

In some embodiments, the treatment of hepatic steatosis comprises a reduction in the amount of hepatic steatosis by about 1% to about 50%, about 25% to about 75%, or about 50% to about 100%. In some embodiments, the treatment of hepatic steatosis comprises a reduction in the amount of hepatic steatosis by about 5%, bout 10%, about 15%, about 20%, about 25%, about 30%, about 35%, about 40%, about 45%, about 50%, about 55%, about 60%, about 65%, about 70%, about 75%, about 80%, about 85%, about 90%, or about 95%.

In some embodiments, (a), (b), and (c) are administered concurrently. In some embodiments, (a), (b), and (c) are administered as a fixed combination. In some embodiments, (a), (b), and (c) are administered as a non-fixed combination. In some embodiments, (a), (b), and (c) are administered sequentially and in any order, at specific or varying time intervals (e.g., during the period of time). In some embodiments, a therapeutically effective amount of each of (a), (b), and (c) are administered concurrently. In some embodiments, a therapeutically effective amount of each of (a), (b), and (c) are administered sequentially and in any order, at specific or varying time intervals (e.g., during the period of time).

Also provided herein are pharmaceutical combinations comprising or consisting essentially of (a) the compound of Formula (I), or a pharmaceutically acceptable salt thereof, (b) an SGLT-2 inhibitor, or a pharmaceutically acceptable salt or solvate thereof, (c) a DPP-4 inhibitor, or a pharmaceutically acceptable salt thereof, and one or more pharmaceutical excipients, for concurrent or sequential administration for use in the treatment of non-alcoholic fatty liver disease (NAFLD). In some embodiments, the pharmaceutical combination further comprises at least one pharmaceutically acceptable carrier.

Also provided herein are pharmaceutical combinations comprising or consisting essentially of (a) the compound of Formula (I), or a pharmaceutically acceptable salt thereof, and (b) an SGLT-2 inhibitor, or a pharmaceutically acceptable salt or solvate thereof, (c) a DPP-4 inhibitor, or a pharmaceutically acceptable salt thereof, and one or more pharmaceutical excipients, for concurrent or sequential administration during a period of time for use in the treatment of non-alcoholic fatty liver disease (NAFLD). In some embodiments, the pharmaceutical combination further comprises at least one pharmaceutically acceptable carrier.

In some embodiments, the pharmaceutical composition is administered to the subject twice a day, daily, every other day, three times a week, twice a week, weekly, every other week, twice a month, or monthly. In some embodiments, the pharmaceutical composition is administered to the subject daily.

In some embodiments, the amount of the compound of Formula (I), or a pharmaceutically acceptable salt thereof, in the composition is from about 0.1 to about 15 milligrams (mg). For example, from about 0.1 to about 10 mg, about 5 to about 15 mg, or about 2 to about 12 mg. In some embodiments, the compound of Formula (I), or a pharmaceutically acceptable salt thereof, in the composition is from about 0.1 to about 5 mg, about 0.1 to about 4 mg, about 0.5 to about 3 mg, about 0.5 to about 2 mg, about 0.5 to about 1 mg, about 1 to about 3 mg, about 2 to about 4 mg, about 3 to about 5 mg, about 1 to about 6 mg, about 2 to about 6 mg, about 3 to about 6 mg, about 4 to about 6 mg, or about 5 to about 6 mg. In some embodiments, the composition comprises a therapeutically effective amount of the compound of Formula (I), or a pharmaceutically acceptable salt thereof.

In some embodiments, the amount of the compound of Formula (I), or a pharmaceutically acceptable salt thereof, in the composition is about 3 mg. In some embodiments, the amount of the compound of Formula (I), or a pharmaceutically acceptable salt thereof, in the composition is about from about 0.1 to about 10.0 mg. In some embodiments, the amount of the compound of Formula (I), or a pharmaceutically acceptable salt thereof, in the composition is about from about 0.1 to about 3 mg. In some embodiments, the amount of the compound of Formula (I), or a pharmaceutically acceptable salt thereof, in the composition is about 0.5 mg. In some embodiments, the amount of the compound of Formula (I), or a pharmaceutically acceptable salt thereof, in the composition is about 1 mg. In some embodiments, the amount of the compound of Formula (I), or a pharmaceutically acceptable salt thereof, in the composition is about 2 mg.

In some embodiments, the compound of Formula (I) is in the form of a besylate salt. In some embodiments, the compound of Formula (I) is in the form of an HCl salt. In some embodiments, the compound of Formula (I) is in the form of an HBr salt. In some embodiments, the compound of Formula (I) is in the form of a tosylate salt.

In some embodiments, the amount of the SGLT-2 inhibitor, or a pharmaceutically acceptable salt or solvate thereof, is from about 1 to about 350 mg. For example, about 1 to about 175 mg, about 175 to about 350 mg, or about 90 to about 260 mg. In some embodiments, the amount of the SGLT-2 inhibitor, or a pharmaceutically acceptable salt or solvate thereof, is from about 85 to about 325 mg. In some embodiments, the amount of the SGLT-2 inhibitor, or a pharmaceutically acceptable salt or solvate thereof, is from about 1 to about 50 mg, about 20 to about 70 mg, about 50 to about 100 mg, about 70 to about 120 mg, about 90 to about 140 mg, about 110 to about 160 mg, about 130 to about 180 mg, about 150 to about 200 mg, about 170 to about 220 mg, about 190 to about 240 mg, about 210 to about 260 mg, about 230 to about 280 mg, about 250 to about 300 mg, about 270 to about 320 mg, or about 290 to about 350 mg. For example, about 100 mg or about 300 mg.

In some embodiments, the SGLT-2 inhibitor is selected from the group consisting of: empagliflozin, canagliflozin, dapagliflozin, ertugliflozin, ipragliflozin, luseogliflozin, remogliflozin etabonate, serfliflozin etabonate, sotagliflozin, tofogliflozin, or a combination of two or more thereof. In some embodiments, the SGLT-2 inhibitor is empagliflozin.

In some embodiments, the SGLT-2 inhibitor is canagliflozin. In some embodiments, the composition comprises 100 mg or 300 mg of canagliflozin. In some embodiments, the SGLT-2 inhibitor is dapagliflozin. In some embodiments, the composition comprises 5 mg or 10 mg of dapagliflozin. In some embodiments, the SGLT-2 inhibitor is empagliflozin. In some embodiments, the composition comprises 10 mg or 25 mg of empagliflozin. In some embodiments, the SGLT-2 inhibitor is ertugliflozin. In some embodiments, the composition comprises 5 mg or 15 mg of ertugliflozin. In some embodiments, the SGLT-2 inhibitor is ipragliflozin. In some embodiments, the composition comprises 25 mg or 50 mg of ipragliflozin. In some embodiments, the SGLT-2 inhibitor is bexagliflozin. In some embodiments, the composition comprises 20 mg of bexagliflozin. In some embodiments, the SGLT-2 inhibitor is sotagliflozin. In some embodiments, the composition comprises 200 mg or 400 mg of sotagliflozin. In some embodiments, the SGLT-2 inhibitor is licogliflozin. In some embodiments, the composition comprises 15 mg, 50 mg, 75 mg or 150 mg of licogliflozin.

In some embodiments, the amount of the DPP-4 inhibitor, or a pharmaceutically acceptable salt thereof, is from about 1 to about 400 mg. For example, about 1 mg to about 20 mg, about 5 mg to about 50 mg, about 25 mg to about 100 mg, about 50 mg to about 200 mg, or about 100 mg to about 400 mg.

In some embodiments, the DPP-4 inhibitor is selected from the group consisting of: sitagliptin, vildagliptin, saxagliptin, linagliptin, gemigliptin, anagliptin, teneligliptin, alogliptin, trelagliptin, omarigliptin, evogliptin, and dutogliptin, or pharmaceutically acceptable salt of any of the foregoing. In some embodiments, the DPP-4 inhibitor is sitagliptin.

In some embodiments, the DPP-4 inhibitor is sitagliptin. In some embodiments, the composition comprises 25 mg, 50 mg, 75 mg, or 100 mg of sitagliptin. In some embodiments, the DPP-4 inhibitor is vildagliptin. In some embodiments, the composition comprises 25 mg, 50 mg, 75 mg, or 100 mg of vildagliptin. In some embodiments, the DPP-4 inhibitor is saxagliptin. In some embodiments, the composition comprises 1 mg, 2 mg, 2.5 mg, or 5 mg of saxagliptin. In some embodiments, the DPP-4 inhibitor is linagliptin. In some embodiments, the composition comprises 1 mg, 2 mg, 2.5 mg, or 5 mg of linagliptin. In some embodiments, the DPP-4 inhibitor is gemigliptin. In some embodiments, the composition comprises 10 mg, 25 mg, or 50 mg of gemigliptin. In some embodiments, the DPP-4 inhibitor is anagliptin. In some embodiments, the composition comprises 50 mg, 100 mg, 150 mg, or 200 mg of anagliptin. In some embodiments, the DPP-4 inhibitor is teneligliptin. In some embodiments, the composition comprises 5 mg, 10 mg, 20 mg, or 40 mg of teneligliptin. In some embodiments, the DPP-4 inhibitor is alogliptin. In some embodiments, the composition comprises 5 mg, 10 mg, 15 mg, or 25 mg of alogliptin. In some embodiments, the DPP-4 inhibitor is trelagliptin. In some embodiments, the composition comprises 10 mg, 25 mg, 50 mg, or 100 mg of trelagliptin. In some embodiments, the DPP-4 inhibitor is omarigliptin. In some embodiments, the composition comprises 5 mg, 10 mg, 15 mg, or 25 mg of omarigliptin. In some embodiments, the DPP-4 inhibitor is evogliptin. In some embodiments, the composition comprises 1 mg, 2.5 mg, 5 mg, or 10 mg of evogliptin. In some embodiments, the DPP-4 inhibitor is dutogliptin. In some embodiments, the composition comprises 50 mg, 100 mg, 200 mg, or 400 mg of dutogliptin.

In some embodiments, the treatment of NAFLD, e.g., NAFL or NASH, comprises a reduction in hepatic steatosis. For example, hepatic steatosis is decreased by at least 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or more than 99% following administration of (a), (b), and (c) for a period of time.

In some embodiments, the treatment of NAFLD, e.g., NAFL or NASH, is assessed using the NAFLD Activity Score (NAS). In some embodiments, treatment of NAFLD comprises a decrease in the NAS. In some embodiments, the NAS for a sample from the subject following administration is 7 or less. In some embodiments, the NAS for a sample from the subject following administration is 5 or less, 4 or less, 3 or less, or 2 or less. In some embodiments, the NAFLD activity score (NAS) for a sample from the subject following administration during the period of time is 7 or less. In some embodiments, the NAS for a sample from the subject following administration during the period of time is 5 or less, 4 or less, 3 or less, or 2 or less. In some embodiments, the sample from the subject is from a liver biopsy.

In some embodiments, the treatment of NAFLD, e.g., NAFL or NASH, can be assessed using the NAFLD Activity Score (NAS). In some embodiments, the NAS for a sample from the subject following administration is reduced by 1 or more, 2 or more, 3 or more, 4 or more, 5 or more, or 6 or more. In some embodiments, the NAS for a sample from the subject following administration is reduced by 1, 2, 3, 4, 5, or 6. In some embodiments, the NAFLD activity score (NAS) for a sample from the subject following administration during the period of time is reduced by 1 or more, 2 or more, 3 or more, 4 or more, 5 or more, or 6 or more. In some embodiments, the NAS for a sample from the subject following administration during the period of time is reduced by 1, 2, 3, 4, 5, or 6. In some embodiments, the sample from the subject is from a liver biopsy.

In some embodiments, the treatment of NAFLD, e.g., NAFL or NASH, comprises treatment of hepatic inflammation. In some embodiments, the severity of the hepatic inflammation is decreased by about 1% to about 50%, about 25% to about 75%, or about 50% to about 100%. In some embodiments, the severity of hepatic inflammation is decreased by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, or about 95%.

In some embodiments, the treatment of NAFLD, e.g., NAFL or NASH, comprises treatment of fibrosis. In some embodiments, the treatment of the NAFLD comprises treatment of cirrhosis (e.g., stage 4 of fibrosis). In some embodiments, treatment of fibrosis comprises a decrease in the stage of fibrosis, for example, from stage 4 to stage 3, from stage 4 to stage 2, from stage 4 to stage 1, from stage 4 to stage 0, from stage 3 to stage 2, from stage 3 to stage 1, from stage 3 to stage 0, from stage 2 to stage 1, from stage 2 to stage 0, or from stage 1 to stage 0.

In some embodiments, the adiponectin level in the subject is increased by at least about 30%, at least about 68%, at least about 175%, or at least about 200%. In some embodiments, the increase is by at least about 175%.

In some embodiments, the level of aspartate aminotransferase (AST) in the subject does not increase. In some embodiments, the level of aspartate aminotransferase (AST) in the subject decreases. In some embodiments, the level of alanine aminotransferase (ALT) in the subject does not increase. In some embodiments, the level of alanine aminotransferase (ALT) in the subject decreases. In some embodiments, the total body weight of the subject does not increase. In some embodiments, the total body weight of the subject decreases. In some embodiments, the body mass index (BMI) of the subject does not increase. In some embodiments, the body mass index (BMI) of the subject decreases. In some embodiments, the waist and hip (WTH) ratio of the subject does not increase. In some embodiments, the waist and hip (WTH) ratio of the subject decreases.

In some embodiments, treatment of NAFLD comprises a decrease in the level of one or more biomarkers indicative of one or more of liver damage, inflammation, fibrosis, and/or cirrhosis, e.g., any of the biomarkers as described herein. In some embodiments, treatment of NAFLD comprises a decrease in the level of one or more biomarkers indicative of one or more of liver damage, inflammation, fibrosis, and/or cirrhosis by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 99%.

In some embodiments, the treatment of NAFLD decreases the level of serum bile acids in the subject. In some embodiments, the treatment of NAFLD comprises treatment of pruritus.

In some embodiments, the subject has liver fibrosis associated with the NAFLD. In some embodiments, the subject has hepatic cirrhosis (e.g., stage 4 fibrosis) associated with the NAFLD. In some embodiments, the subject has liver fibrosis as a comorbidity. In some embodiments, the subject has hepatic cirrhosis (e.g., stage 4 fibrosis) as a comorbidity. In some embodiments, the subject has liver fibrosis caused by the NAFLD. In some embodiments, the subject has hepatic cirrhosis (e.g., stage 4 fibrosis) caused by the NAFLD.

In some embodiments, the NAFLD is simple nonalcoholic fatty liver (NAFL). In some embodiments, the NAFLD is NAFL with attendant liver fibrosis. In some embodiments, the NAFLD is NAFL with attendant liver cirrhosis.

In some embodiments, the NAFLD is nonalcoholic steatohepatitis (NASH). In some embodiments, the NAFLD is NASH with attendant liver fibrosis. In some embodiments, the NAFLD is NASH with attendant liver cirrhosis.

In some embodiments, the method further comprises performing a liver biopsy to determine the NAFLD activity score of the biopsy sample obtained from the subject.

In some embodiments, (a), (b), and (c) are administered prophylactically.

In some embodiments, the subject was previously treated, before the period of time, with one or more therapeutic agents, e.g., treatment with at least one NAFLD treatment. In some embodiments, the one or more therapeutic agents that were administered to the subject before the period of time was unsuccessful (e.g., therapeutically unsuccessful as determined by a physician). In some embodiments, the unsuccessful treatment did not comprises or consist essentially of administration of (a), (b), and (c).

### EXAMPLES

The following example further illustrates the invention. For example, the efficacy of CHS-131, alone or in combination with other therapeutic agents, to treat NAFLD is determined in the following example.

### Example 1

This study assesses the effects of treatment with CHS-131 (Compound of Formula (I)), alone and in combination with other therapeutic agents, to treat NASH. Metabolic parameters, hepatic pathology, and NAFLD Activity Score including fibrosis stage are evaluated in male DIO-NASH mice.

In particular this study will permit mechanistic evaluation of the effects of CHS-131, an SGTL2-inhibitor (empagliflozin), and a DPP-4 inhibitor (sitagliptin) monotherapy, and CH-131+an SGTL2-inhibitor (empagliflozin), +a DPP-4 inhibitor (sitagliptin) combination therapy on the NASH disease process. For example, gene expression levels are measured various tissues (as described herein), including FAS, ACC, Srebp-1c, Srebp2, PPARγ, Pepck, aP2, Cidea, Cidec, and adiponectin in mice not receiving a treatment or receiving vehicle, and mice that have received a treatment. Similarly, mitochondrial and peroxisomal β-oxidation will be indirectly assessed by measuring gene expression levels of Cpt1α, Cpt1β, Vicad, Acox1, Dbp1, Mcad1, and Pdk4 in mice not receiving a treatment or receiving a placebo, and mice that receiving a treatment. Chromatography and mass spectrometric analysis will be used to assess the presence and relative amounts fatty acids in liver tissue, along with the presence of particular lipid/fatty acid metabolites and other lipid molecules, such as ceramides, diacyglycerol, lysophosphatidylcholine, and lipotoxic lipids, in mice not receiving a treatment or receiving vehicle, and mice receiving a treatment. Other cellular pathways may also be assessed, including those involved in apoptosis, necrosis, and inflammation in mice not receiving a treatment or receiving vehicle, and mice receiving a treatment. For example, cytokine gene expression such as TNFα, Ccl3, Cxcl10, IL-1β, IL-6, and Mcp-1; and expression of M1 and M2 macrophage markers such as Cd11b, Cd11c, CD163, CD206, and Ym1/2 can be assessed in mice not receiving a treatment or vehicle, and mice receiving a treatment.

Additional mechanistic evaluation into the molecular basis for the effects of CHS-131, an SGTL2-inhibitor (empagliflozin), and a DPP-4 inhibitor (sitagliptin) monotherapy, and CH-131+an SGTL2-inhibitor (empagliflozin)+a DPP-4 inhibitor (sitagliptin) combination therapy on the NASH disease process include determining expression levels (e.g., protein and/or mRNA) of hepatic stellate cell activation and liver fibrosis (such as Tgfb1 and Fn1) and hepatic signaling such as expression and phosphorylation levels of proteins including AKT, AMPK, JNK, STAT3 and SOCS1 in mice not receiving a treatment or receiving vehicle, and mice receiving a treatment.

This example also includes evaluating the effects of CHS-131, an SGTL2-inhibitor (empagliflozin), and a DPP-4 inhibitor (sitagliptin) monotherapy, and CH-131+an SGTL2-inhibitor (empagliflozin)+a DPP-4 inhibitor (sitagliptin) combination therapy on pathways involved in in hepatic insulin resistance and NAFLD in mice not receiving a treatment or receiving vehicle, and mice receiving a treatment. For example, determining adipose tissue morphology and adipocyte size via IHC, and evaluating fatty acid metabolism in visceral and subcutaneous adipose tissue in mice not receiving a treatment or receiving a vehicle, and mice receiving a treatment. These effects can also be determined by assessing the expression levels (e.g., protein and/or mRNA) of UCP1, CIDEA, ELVOL3, PRDM16, PGC-1α, aP2, PPARγ, Cd36, Hsl, Atgl, CPT1β, mtTFA, mtCOX2, and Cytc in mice not receiving a treatment or receiving a vehicle, and mice receiving a treatment.

Expression levels of cytokines, chemokines, and M1 and M2 macrophage markers will also be determined in the context of in hepatic insulin resistance and NAFLD, for example, levels of TNFα, IL-6, IL-8, MCP-1, Cd11c, CD163, CD206, and Ym1/2 in mice not receiving a treatment or receiving vehicle, or mice receiving a treatment. Adipokine and hormone expression levels may also be measured with various immunoassays, including levels of leptin and adiponectin, in mice not receiving a treatment or receiving vehicle, and mice receiving a treatment.

Measurements in peripheral tissues such as serum, whole blood, or plasma, may also be performed in mice not receiving a treatment or receiving vehicle, and mice receiving a treatment. For example, assessing the lipid profiles in these tissues, via chromatography, such as LDL, VLDL, HDL, total cholesterol, and triglycerides. An overview of the study is provided in Table 3, below.

TABLE 3

| | | | | | Dosing | | | |
|---|---|---|---|---|---|---|---|---|
| | | | | | | | | Time |
| | | Mouse | | Dose | | | | of |
| Group | Compound | Model | n | (mg/kg) | Method | Volume | Frequency | Day |
| 1 | Chow Vehicle + Vehicle | LEAN- | 12 | NA | PO | 5 + 5 | QD | AM |
| | | CHOW | | | | | | |
| 2 | NASH Vehicle + Vehicle | DIO- | 12 to 14 | NA | PO | 5 + 5 | QD | AM |
| | | NASH | | | | | | |
| 3 | Low dose Compound of | DIO- | 12 to 14 | 10 | PO | 5 + 5 | QD | AM |
| | Formula (I) + Vehicle | NASH | | | | | | |
| 4 | High dose Compound of | DIO- | 12 to 14 | 30 | PO | 5 + 5 | QD | AM |
| | Formula (I) + Vehicle | NASH | | | | | | |
| 5 | Empagliflozin + Vehicle | DIO- | 12 to 14 | 10 | PO | 5 + 5 | QD | AM |
| | | NASH | | | | | | |
| 6 | Sitagliptin + Vehicle | DIO- | 12 to 14 | 30 + 10 | PO | 5 + 5 | QD | AM |
| | | NASH | | | | | | |
| 7 | Low dose Compound of | DIO- | 12 to 14 | 0.4 | SC + PO | 5 + 5 | QD | AM |
| | Formula (I) + | NASH | | | | | | |
| | Empagliflozin + | | | | | | | |
| | Sitagliptin | | | | | | | |
| 8 | High dose Compound of | DIO- | 12 to 14 | 30 + 0.4 | PO + SC | 5 + 5 | QD | AM |
| | Formula (I) + | NASH | | | | | | |
| | Empagliflozin + | | | | | | | |
| | Sitagliptin | | | | | | | |
| 9 | Elafibranor + | DIO- | 12 to 14 | 30 | PO | 5 + 5 | QD | AM |
| | Vehicle | NASH | | | | | | |

PO is per oral; SC is subcutaneous; QD is once a day. Groups 2-6 are fed a HF-HD diet.

Each animal is administered the respective compositions starting on Day 0 and ending on Day 82-84. The compositions are as described in Table 4.

TABLE 4
Compositions

| Name | Dissolved In | Process |
|---|---|---|
| Vehicle | 1% methyl cellulose in | N/A |
| | deionized water | |
| CHS-131 | 1% methyl cellulose in | Stir for 30-60 minutes before |
| | deionized water | and during dosing |
| Empagliflozin | 1% methyl cellulose in | Dissolved |
| | deionized water | |
| Sitagliptin | 1% methyl cellulose in | Dissolved |
| | deionized water | |
| Elafibranor | 1% methyl cellulose in | Mix |
| | deionized water | |

Samples, as described in Table 5, are collected before, during, and after the study.

TABLE 5
Samples collected over course of study

| Sample | Usage | Groups | Time Point | Method |
|---|---|---|---|---|
| Liver pre-biopsy | Stratification and | All | 3 weeks | Dissection |
| | randomization, NAFLD | | before start | |
| | Activity Score, Fibrosis | | of study | |
| | Stage, Col1a1 | | | |
| BG Baseline | Blood Glucose | All | 1 week | Tail Vein |
| | | | before start | |
| | | | of study | |
| Plasma insulin baseline | Plasma insulin | All | 1 week | Tail Vein |
| | | | before start | |
| | | | of study | |
| OGTT | Blood Glucose | All | Week 7-8 | Tail Vein |
| IPTT | Blood Glucose | All | Week 9/10 | Tail Vein |
| BG week 12 | Blood Glucose | All | Week 12 | Tail Vein |
| Plasma insulin week 12 | Plasma insulin | All | Week 12 | Tail Vein |
| Terminal | ALT/AST/TG/TC/BUN | All | Week 12 | Tail Vein |
| ALT/AST/TG/ | | | | |
| TC/BUN/creatinine | | | | |
| Liver post-biopsy | NAFLD Activity Score, | All | Termination | Dissection |
| | and fibrosis stage and | | | |
| | steatosis stage and Col1a1 | | | |
| | and Galectin-3 and a-SMA | | | |
| | quantification | | | |
| Liver TG/TC | Liver triglyceride and total | All | Termination | Dissection |
| | cholesterol | | | |
| Liver HP | Liver hydroxyproline | All | Termination | Dissection |
| Liver RNA | RNAseq (optional) | All | Termination | Dissection |
| Liver | Evaluation | All | Termination | Dissection |
| Muscle | Evaluation | All | Termination | Dissection |
| Epididymal fat | Evaluation | All | Termination | Dissection |
| Subcutaneous fat | Evaluation | All | Termination | Dissection |
| Kidney | Evaluation | All | Termination | Dissection |
| Brain | Evaluation | All | Termination | Dissection |
| Heart | Evaluation | All | Termination | Dissection |
| Terminal plasma | Evaluation | All | Termination | Cardiac |
| | | | | Puncture |

ALT is alanine transaminase; a-SMA is alpha-smooth muscle actin; AST is aspartate transaminase; BG is blood glucose; BUN is blood urea nitrogen; Col1a1 is collagen 1a1; OGTT is oral glucose tolerance test; IPITT is intraperitoneal insulin tolerance test; TG is triglycerides; TC is total cholesterol; HP is hydroxyproline

An overview of sample analyses that are performed during the study are listed in Tables 6-8, below.

TABLE 6
In vivo pharmacology

| Analysis | | | | |
|---|---|---|---|---|
| Name | Groups | Samples | Period or Frequency | Comments |
| Bodyweight | All | Whole | QD | Na |
| | | animal | | |
| Food intake | All | Whole | QD week 0 + 1 | AM |
| | | animal | QW (24 h) Week 2-12 | |
| Echo MRI | All | Whole | Week 1 | Na |
| baseline | | animal | | |
| Echo MRI | All | Whole | Week 11 | Na |
| week 11 | | animal | | |
| Liver | All | Whole liver | Termination | Na |
| weight | | weight (wet | | |
| | | weight) | | |

TABLE 7
Histology

| Analysis Name | Groups | Samples | Comments |
|---|---|---|---|
| Fibrosis stage | All | Liver pre-biopsy | PSR staining |
| | | Liver post-biopsy | Re-staining of |
| | | | pre-biopsy |
| NAFLD Activity | All | Liver pre-biopsy | HE staining |
| Score | | Liver post-biopsy | Re-staining of |
| | | | pre-biopsy |
| Col1a1 | All | Liver pre-biopsy | IHC (randomization) |
| | | Liver post-biopsy | IHC |
| Galectin-3 | All | Liver post-biopsy | IHC |
| quantification | | | |
| Steatosis | All | Liver post-biopsy | HE staining |
| quantification | | | |
| a-SMA | All | Liver post-biopsy | IHC |
| quantification | | | |

TABLE 8
Assays

| Analysis Name | Groups | Samples |
|---|---|---|
| Plasma insulin | All | Plasma insulin baseline |
| baseline | | |
| Plasma insulin | All | Plasma insulin baseline |
| week 12 | | |
| Plasma ALT | All | Terminal ALT/AST/TG/TC/BUN/Creatinine |
| Plasma AST | All | Terminal ALT/AST/TG/TC/BUN/Creatinine |
| Plasma TG | All | Terminal ALT/AST/TG/TC/BUN/Creatinine |
| Plasma TC | All | Terminal ALT/AST/TG/TC/BUN/Creatinine |
| Plasma BUN | All | Terminal ALT/AST/TG/TC/BUN/Creatinine |
| Plasma creatinine | All | Terminal ALT/AST/TG/TC/BUN/Creatinine |
| Liver | All | Liver TG/TC |
| triglycerides | | |
| Liver total | All | Liver TG/TC |
| cholesterol | | |
| Liver | All | Liver HP |
| hydroxyproline | | |

NAFLD Activity Score (NAS) and Fibrosis stage are evaluated as follows. Liver samples are fixed in formalin, paraffin embedded and sections are stained with hematoxylin and eosin (H&E) and Sirius Red. Samples are scored for NAS and fibrosis stage (outlined below) using of the clinical criteria outlined by Kleiner et al. 2005. Total NAS score represents the sum of scores for steatosis, inflammation, and ballooning, and ranges from 0-8.

TABLE 9
Total NAS scoring

| Feature | Degree | Score |
|---|---|---|
| Steatosis | <5% | 0 |
| | 5-33% | 1 |
| | >33-66% | 2 |
| | >66% | 3 |
| Lobular inflammation | No foci | 0 |
| | <2 foci/200x | 1 |
| | 2-4 foci/200x | 2 |
| | >4 foci/200x | 3 |
| Ballooning degeneration | None | 0 |
| | Few | 1 |
| | Many cells/prominent ballooning | 2 |
| Fibrosis | None | 0 |
| | Perisinusoidal or periportal | 1 |
| | Perisinusoidal & portal/periportal | 2 |
| | Bridging fibrosis | 3 |
| | Cirrhosis | 4 |

Adopted from: Design and validation of a histological scoring system for nonalcoholic fatty liver disease, Kleiner et al., Hepatology 41; 2005.

For lobular inflammation, inflammation is evaluated by counting the number of inflammatory foci per field using a 200× magnification (min. 5 fields per animal). A focus is defined as a cluster, not a row, of >3 inflammatory cells. Acidophil bodies are not included in this assessment, nor is portal inflammation. Fibrosis stage is evaluated separately from NAS.

### IHC and Steatosis Quantification

Quantitative assessment of immunoreactivity is evaluated as follows. IHC-positive staining is quantified by image analysis using the Visiomorph software (Visiopharm, Denmark). Visiomorph protocols are designed to analyze the virtual slides in two steps: 1. Crude detection of tissue at low magnification (1×objective). The liver capsule is excluded. 2. Detection of IHC-positive staining (e.g. green; collagen 1 IHC), tissue (e.g. red) and fat (e.g. pink) at high magnification (10×objective). The quantitative estimate of IHC-positive staining is calculated as an area fraction (AF) according to the following formula:

$$A {F}_{IHC - pos} = \frac{{Area}_{IHC - pos .}}{{Area}_{f a t} + {Area}_{t i s s u e} + {Area}_{IHC - pos}}$$

Quantitative assessment of steatosis is evaluated as follows. Steatosis is quantified on H&E stained slides by image analysis using the Visiomorph software (Visiopharm, Denmark). Visiomorph protocols are designed to analyze the virtual slides in two steps: 1. Crude detection of tissue at low magnification (1×objective). 2. Detection of steatosis (pink) and tissue (blue) at high magnification (20×objective). The quantitative estimate of steatosis is calculated as an area fraction (AF) according to the following formula:

$$A {F}_{s t e a t o s i s} = \frac{{Area}_{s t e a t o s i s}}{{Area}_{t i s s u e} + {Area}_{s t e a t o s i s}}$$

What is claimed is:

1. A method of treating non-alcoholic fatty liver disease (NAFLD) in a subject in need thereof comprising administering to the subject
(a) the compound of Formula (I), 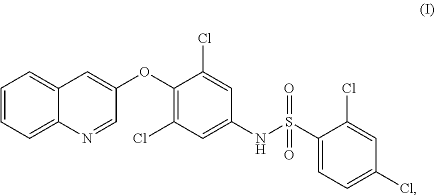 [SMILES: O=S(=O)(Nc1cc(Cl)c(Oc2cnc3ccccc3c2)c(Cl)c1)c1ccc(Cl)cc1Cl]
or a pharmaceutically acceptable salt thereof,
(b) an SGLT inhibitor, or a pharmaceutically acceptable salt or solvate thereof, and
(c) a DPP-4 inhibitor, or a pharmaceutically acceptable salt thereof;
wherein the amounts of (a), (b), and (c) together are effective in treating NAFLD.

2. A method of treating a subject, the method comprising:
selecting a subject having non-alcoholic fatty liver disease (NAFLD); and
administering
(a) the compound of Formula (I), 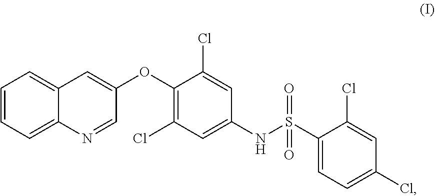 [SMILES: O=S(=O)(Nc1cc(Cl)c(Oc2cnc3ccccc3c2)c(Cl)c1)c1ccc(Cl)cc1Cl]
or a pharmaceutically acceptable salt thereof,
(b) an SGLT inhibitor, or a pharmaceutically acceptable salt or solvate thereof, and
(c) a DPP-4 inhibitor, or a pharmaceutically acceptable salt thereof;
wherein the amounts of (a), (b), and (c) together are effective in treating NAFLD.

3. A method of treating a subject, the method comprising:
identifying a subject having non-alcoholic fatty liver disease (NAFLD); and
administering
(a) the compound of Formula (I), 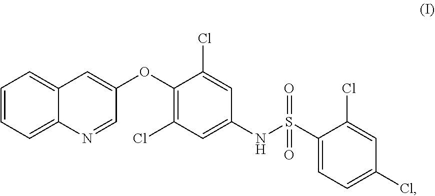 [SMILES: O=S(=O)(Nc1cc(Cl)c(Oc2cnc3ccccc3c2)c(Cl)c1)c1ccc(Cl)cc1Cl]
or a pharmaceutically acceptable salt thereof,
(b) an SGLT inhibitor, or a pharmaceutically acceptable salt or solvate thereof, and
(c) a DPP-4 inhibitor, or a pharmaceutically acceptable salt thereof
wherein the amounts of (a), (b), and (c) together are effective in treating NAFLD.

4. A method of treating non-alcoholic fatty liver disease (NAFLD) in a subject in need thereof comprising administering to the subject:
(a) a therapeutically effective amount of the compound of Formula (I), 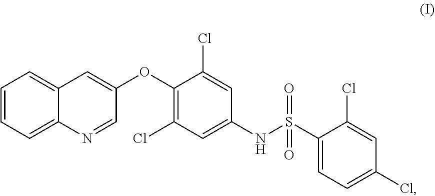 [SMILES: O=S(=O)(Nc1cc(Cl)c(Oc2cnc3ccccc3c2)c(Cl)c1)c1ccc(Cl)cc1Cl]
or a pharmaceutically acceptable salt thereof, and
(b) a therapeutically effective amount of an SGLT inhibitor, or a pharmaceutically acceptable salt or solvate thereof, and
(c) a therapeutically effective amount of a DPP-4 inhibitor, or a pharmaceutically acceptable salt thereof.

5. A method of treating a subject, the method comprising:
selecting a subject having non-alcoholic fatty liver disease (NAFLD); and
administering
(a) a therapeutically effective amount of the compound of Formula (I), 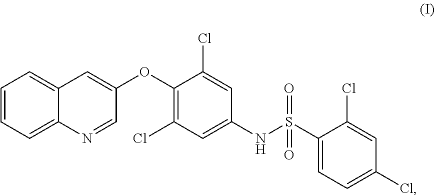 [SMILES: O=S(=O)(Nc1cc(Cl)c(Oc2cnc3ccccc3c2)c(Cl)c1)c1ccc(Cl)cc1Cl]
or a pharmaceutically acceptable salt thereof, and
(b) a therapeutically effective amount of an SGLT inhibitor, or a pharmaceutically acceptable salt or solvate thereof, and
(c) a therapeutically effective amount of a DPP-4 inhibitor, or a pharmaceutically acceptable salt thereof, to the selected subject.

6. A method of treating a subject, the method comprising:
selecting a subject having non-alcoholic fatty liver disease (NAFLD); and
administering
(a) the compound of Formula (I), 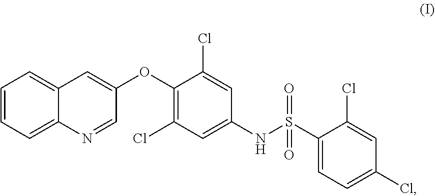 [SMILES: O=S(=O)(Nc1cc(Cl)c(Oc2cnc3ccccc3c2)c(Cl)c1)c1ccc(Cl)cc1Cl]
or a pharmaceutically acceptable salt thereof,
(b) an SGLT inhibitor, or a pharmaceutically acceptable salt or solvate thereof, and
(c) a DPP-4 inhibitor, or a pharmaceutically acceptable salt thereof, to the selected subject;
wherein the amounts of (a), (b), and (c) together are effective in treating NAFLD.

7. A method of treating a subject, the method comprising:
identifying a subject having non-alcoholic fatty liver disease (NAFLD); and
administering
(a) the compound of Formula (I), 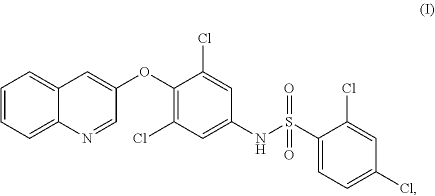 [SMILES: O=S(=O)(Nc1cc(Cl)c(Oc2cnc3ccccc3c2)c(Cl)c1)c1ccc(Cl)cc1Cl]
or a pharmaceutically acceptable salt thereof,
(b) an SGLT inhibitor, or a pharmaceutically acceptable salt or solvate thereof, and
(c) a DPP-4 inhibitor, or a pharmaceutically acceptable salt thereof, to the subject;
wherein the amounts of (a), (b), and (c) together are effective in treating NAFLD.

8. A method of treating non-alcoholic fatty liver disease (NAFLD) in a subject in need thereof comprising administering to the subject:
(a) a therapeutically effective amount of the compound of Formula (I), 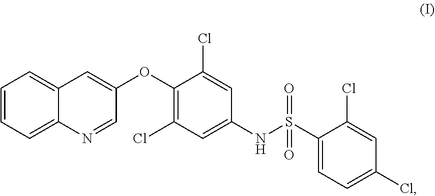 [SMILES: O=S(=O)(Nc1cc(Cl)c(Oc2cnc3ccccc3c2)c(Cl)c1)c1ccc(Cl)cc1Cl]
or a pharmaceutically acceptable salt thereof,
(b) a therapeutically effective amount of an SGLT inhibitor, or a pharmaceutically acceptable salt or solvate thereof, and
(c) a therapeutically effective amount of a DPP-4 inhibitor, or a pharmaceutically acceptable salt thereof.

9. A method of treating a subject, the method comprising:
selecting a subject having non-alcoholic fatty liver disease (NAFLD); and
administering
(a) a therapeutically effective amount of the compound of Formula (I), 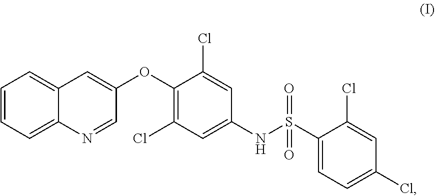 [SMILES: O=S(=O)(Nc1cc(Cl)c(Oc2cnc3ccccc3c2)c(Cl)c1)c1ccc(Cl)cc1Cl]
or a pharmaceutically acceptable salt thereof,
(b) a therapeutically effective amount of an SGLT inhibitor, or a pharmaceutically acceptable salt or solvate thereof, and
(c) a therapeutically effective amount of a DPP-4 inhibitor, or a pharmaceutically acceptable salt thereof, to the selected subject.

10. The method of any one of claims 1-9, wherein (a), (b), and (c) are administered concurrently.

11. The method of any one of claims 1-9, wherein (a), (b), and (c) are administered sequentially in any order.

12. The method of any one of claims 1-11, wherein the treatment of NAFLD comprises a reduction in hepatic steatosis.

13. The method of any one of claims 1-12, wherein the treatment of NAFLD comprises a reduction in hepatic inflammation.

14. The method of any one of claims 1-13, wherein the NAFLD activity score (NAS) following administration is 7 or less.

15. The method of any one of claims 1-14, wherein the NAS is 5 or less.

16. The method of any one of claims 1-15, wherein the NAS is 3 or less.

17. The method of any one of claims 1-16, wherein the treatment of the NAFLD comprises treatment of liver cirrhosis.

18. The method of any one of claims 1-17, wherein the adiponectin level in the subject is increased by at least about 30%, at least about 68%, at least about 175%, or at least about 200%.

19. The method of any one of claims 1-18, wherein the level of one or more biomarkers indicative of one or more of liver damage, inflammation, fibrosis, and/or cirrhosis is decreased.

20. The method of claim 19, wherein the increase is by at least about 175%.

21. The method of any one of claims 1-20, wherein the treatment of NAFLD comprises treatment of pruritus.

22. The method of any one of claims 1-21, wherein the subject has hepatic cirrhosis associated with the NAFLD.

23. The method of any one of claims 1-22, wherein the subject has hepatic cirrhosis as a comorbidity.

24. The method of any one of claims 1-22, wherein the subject has hepatic cirrhosis caused by the NAFLD.

25. The method of any one of claims 1-22, wherein the NAFLD is NAFL with attendant liver cirrhosis.

26. The method of any one of claims 1-25, wherein the treatment of NAFL decreases the level of serum bile acids in the subject.

27. The method of any one of claims 1-26, wherein the NAFLD is nonalcoholic steatohepatitis (NASH).

28. The method of any one of claims 1-27, wherein the NAFLD is NASH with attendant liver cirrhosis.

29. The method of any one of claims 27-28, wherein the treatment of NASH decreases the level of serum bile acids in the subject.

30. The method of any one of claims 27-29, wherein the treatment of NASH comprises treatment of pruritus.

31. The method of any one of claims 1-27, wherein the NAFLD is simple nonalcoholic fatty liver (NAFL).

32. The method of any one of claims 1-31, wherein the method further comprises performing a liver biopsy to determine the NAFLD activity score of the biopsy sample obtained from the subject.

33. A method of treating fibrosis in a subject in need thereof comprising administering to the subject:
(a) the compound of Formula (I), 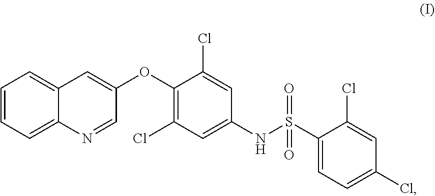 [SMILES: O=S(=O)(Nc1cc(Cl)c(Oc2cnc3ccccc3c2)c(Cl)c1)c1ccc(Cl)cc1Cl]
or a pharmaceutically acceptable salt thereof,
(b) an SGLT inhibitor, or a pharmaceutically acceptable salt or solvate thereof, and
(c) a DPP-4 inhibitor, or a pharmaceutically acceptable salt thereof;
wherein the amounts of (a), (b), and (c) together are effective in treating fibrosis.

34. A method of treating fibrosis in a subject in need thereof comprising administering to the subject:
(a) a therapeutically effective amount of the compound of Formula (I), 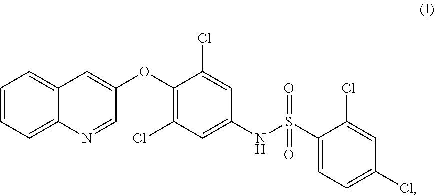 [SMILES: O=S(=O)(Nc1cc(Cl)c(Oc2cnc3ccccc3c2)c(Cl)c1)c1ccc(Cl)cc1Cl]
or a pharmaceutically acceptable salt thereof,
(b) a therapeutically effective amount of an SGLT inhibitor, or a pharmaceutically acceptable salt or solvate thereof, and
(c) a therapeutically effective amount of a DPP-4 inhibitor, or a pharmaceutically acceptable salt thereof.

35. A method of treating fibrosis in a subject in need thereof comprising administering to the subject:
(a) a therapeutically effective amount of the compound of Formula (I), 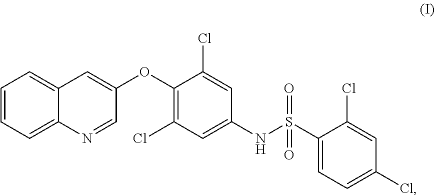 [SMILES: O=S(=O)(Nc1cc(Cl)c(Oc2cnc3ccccc3c2)c(Cl)c1)c1ccc(Cl)cc1Cl]
or a pharmaceutically acceptable salt thereof,
(b) a therapeutically effective amount of an SGLT inhibitor, or a pharmaceutically acceptable salt or solvate thereof, and
(c) a therapeutically effective amount of a DPP-4 inhibitor, or a pharmaceutically acceptable salt thereof;
wherein the amounts of (a), (b), and (c) together are effective in treating fibrosis.

36. The method of any one of claims 33-35, wherein the fibrosis is cirrhosis.

37. The method of any one of claims 33-36, wherein the fibrosis is associated with NAFLD.

38. The method of any one of claims 33-37, wherein the fibrosis is caused by NAFLD.

39. The method of claim 37 or 38, wherein the NAFLD is NASH.

40. The method of any one of claims 37-39, wherein the method further comprises performing a liver biopsy to determine the NAFLD activity score of the biopsy sample obtained from the subject.

41. The method of any one of claims 33-40, wherein the treatment of fibrosis comprises a decrease in the stage of fibrosis, a lack of progression of the fibrosis, or a slowing in the progression of the fibrosis.

42. The method of any one of claims 33-41, wherein the treatment of fibrosis comprises a decrease in the stage of fibrosis.

43. The method of any one of claims 33-42, wherein the decrease in the stage of fibrosis is from stage 4 to stage 3, from stage 4 to stage 2, from stage 4 to stage 1, from stage 4 to stage 0, from stage 3 to stage 2, from stage 3 to stage 1, from stage 3 to stage 0, from stage 2 to stage 1, from stage 2 to stage 0, or from stage 1 to stage 0.

44. The method of any one of claims 1-43, wherein the SGLT-2 inhibitor, or a pharmaceutically acceptable salt or solvate thereof, is selected from the group consisting of: empagliflozin, canagliflozin, dapagliflozin, ertugliflozin, ipragliflozin, luseogliflozin, remogliflozin etabonate, serfliflozin etabonate, sotagliflozin, tofogliflozin, or a pharmaceutically acceptable salt or solvate of any of the foregoing.

45. The method of any one of claims 1 to 44, wherein the SGLT-2 inhibitor, or a pharmaceutically acceptable salt or solvate thereof, is empagliflozin.

46. The method of any one of claims 1 to 45, wherein the SGLT-2 inhibitor, or a pharmaceutically acceptable salt or solvate thereof, is administered at a dose from about 1 to about 350 mg.

47. The method of any one of claims 1 to 46, wherein the SGLT-2 inhibitor, or a pharmaceutically acceptable salt or solvate thereof, is administered at a dose from about 85 to about 325 mg.

48. The method of any one of claims 1 to 47, wherein the SGLT-2 inhibitor, or a pharmaceutically acceptable salt or solvate thereof, is administered at a dose from about 5 to about 15 mg.

49. The method of any one of claims 1 to 48, wherein the SGLT-2 inhibitor, or a pharmaceutically acceptable salt or solvate thereof, is administered at a dose of about 10 mg.

50. The method of any one of claims 1 to 48, wherein the SGLT-2 inhibitor, or a pharmaceutically acceptable salt or solvate thereof, is administered at a dose of about 8 mg.

51. The method of any one of claims 1 to 48, wherein the SGLT-2 inhibitor, or a pharmaceutically acceptable salt or solvate thereof, is administered at a dose of about 5 mg.

52. The method of any one of claims 1 to 51, wherein the SGLT-2 inhibitor, or a pharmaceutically acceptable salt or solvate thereof, is administered to the subject twice a day, daily, every other day, three times a week, twice a week, weekly, every other week, twice a month, or monthly.

53. The method of any one of claims 1 to 52, wherein the SGLT-2 inhibitor, or a pharmaceutically acceptable salt or solvate thereof, is administered to the subject daily.

54. The method of any one of claims 1-53, wherein the DPP-4 inhibitor is selected from the group consisting of: sitagliptin, vildagliptin, saxagliptin, linagliptin, gemigliptin, anagliptin, teneligliptin, alogliptin, trelagliptin, omarigliptin, evogliptin, and dutogliptin, or a pharmaceutically acceptable salt of any of the foregoing.

55. The method of any one of claims 1-54, wherein the DPP-4 inhibitor is sitagliptin.

56. The method of any one of claims 1-55, wherein the DPP-4 inhibitor, or a pharmaceutically acceptable salt thereof, is administered at a dose of about 1 mg to about 100 mg.

57. The method of any one of claims 1-56, wherein the DPP-4 inhibitor, or a pharmaceutically acceptable salt thereof, is administered at a dose of about 5 mg to about 50 mg.

58. The method of any one of claims 1-57, wherein the DPP-4 inhibitor, or a pharmaceutically acceptable salt thereof, is administered at a dose of about 10 mg to about 25 mg.

59. The method of any one of claims 1-56, wherein the DPP-4 inhibitor, or a pharmaceutically acceptable salt thereof, is administered at a dose of about 25 mg to about 75 mg.

60. The method of any one of claims 1-56, wherein the DPP-4 inhibitor, or a pharmaceutically acceptable salt thereof, is administered at a dose of about 50 mg to about 100 mg.

61. The method of any one of claims 1 to 60, wherein the DPP-4 inhibitor, or a pharmaceutically acceptable salt thereof, is administered to the subject twice a day, daily, every other day, three times a week, twice a week, weekly, every other week, twice a month, or monthly.

62. The method of any one of claims 1 to 61, wherein the DPP-4 inhibitor, or a pharmaceutically acceptable salt thereof, is administered to the subject daily.

63. The method of any one of claims 1-62, wherein the compound of Formula (I), a pharmaceutically acceptable salt thereof, is administered prophylactically.

64. The method of any one of claims 1-63, wherein the compound of Formula (I), or a pharmaceutically acceptable salt thereof, is administered at a dose from about 0.1 to about 15 mg.

65. The method of any one of claims 1-64, wherein the compound of Formula (I), or a pharmaceutically acceptable salt thereof, is administered at a dose from about 1 to about 10 mg.

66. The method of any one of claims 1-65, wherein the compound of Formula (I), or a pharmaceutically acceptable salt thereof, is administered at a dose from about 2 to about 6 mg.

67. The method of any one of claims 1-65, wherein the compound of Formula (I), or a pharmaceutically acceptable salt thereof, is administered at a dose from about 0.5 to about 3 mg.

68. The method of any one of claims 1-67, wherein the compound of Formula (I), or a pharmaceutically acceptable salt thereof, is administered at a dose of about 3 mg.

69. The method of any one of claims 1-67, wherein the compound of Formula (I), or a pharmaceutically acceptable salt thereof, is administered at a dose of about 2 mg.

70. The method of any one of claims 1-67, wherein the compound of Formula (I), or a pharmaceutically acceptable salt thereof, is administered at a dose of about 1 mg.

71. The method of any one of claims 1-70, wherein the compound of Formula (I), or a pharmaceutically acceptable salt thereof, is administered to the subject twice a day, daily, every other day, three times a week, twice a week, weekly, every other week, twice a month, or monthly.

72. The method of any one of claims 1-71, wherein the compound of Formula (I), or a pharmaceutically acceptable salt thereof, is administered to the subject daily.

73. The method of any one of claims 1-68, wherein the compound of Formula (I), or a pharmaceutically acceptable salt thereof, is administered to the subject daily and the dose of the compound of Formula (I) is about 3 mg.

74. The method of any one of claims 1-63, wherein the compound of Formula (I), or a pharmaceutically acceptable salt thereof, is administered at a dose from about 0.1-about 10.0 mg per day.

75. The method of any one of claims 1-63, wherein the compound of Formula (I), or a pharmaceutically acceptable salt thereof, is administered at a dose from about 0.1-about 3 mg per day.

76. The method of any one of claims 1-63, wherein the compound of Formula (I), or a pharmaceutically acceptable salt thereof, is administered at a dose of about 0.5 milligram per day.

77. The method of any one of claims 1-63, wherein the compound of Formula (I), or a pharmaceutically acceptable salt thereof, is administered at a dose of about 1 milligram per day.

78. The method of any one of claims 1-63, wherein the compound of Formula (I), or a pharmaceutically acceptable salt thereof, is administered at a dose of about 2 mg per day.

79. The method of any one of claims 1-78, wherein the compound of Formula (I) is in the form of a besylate salt.

80. A pharmaceutical composition comprising
(a) the compound of Formula (I), 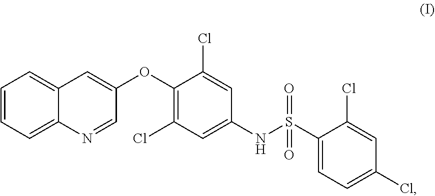 [SMILES: O=S(=O)(Nc1cc(Cl)c(Oc2cnc3ccccc3c2)c(Cl)c1)c1ccc(Cl)cc1Cl]
or a pharmaceutically acceptable salt thereof,
(b) an SGLT-2 inhibitor, or a pharmaceutically acceptable salt or solvate thereof,
(c) a DPP-4 inhibitor, or a pharmaceutically acceptable salt thereof, and
one or more pharmaceutical excipients,
wherein the amounts of (a), (b), and (c) together are effective in treating NAFLD.

81. The pharmaceutical composition of claim 80, wherein the SGLT-2 inhibitor, or a pharmaceutically acceptable salt or solvate thereof, is selected from the group consisting of: empagliflozin, canagliflozin, dapagliflozin, ertugliflozin, ipragliflozin, luseogliflozin, remogliflozin etabonate, serfliflozin etabonate, sotagliflozin, tofogliflozin, or a pharmaceutically acceptable salt or solvate of any of the foregoing.

82. The pharmaceutical composition of claim 80 or 81, wherein the DPP-4 inhibitor, or a pharmaceutically acceptable salt thereof, is selected from the group consisting of: sitagliptin, vildagliptin, saxagliptin, linagliptin, gemigliptin, anagliptin, teneligliptin, alogliptin, trelagliptin, omarigliptin, evogliptin, and dutogliptin, or a pharmaceutically acceptable salt of any of the foregoing.

83. The pharmaceutical composition of any one of claims 80-82, wherein the compound of Formula (I), or a pharmaceutically acceptable salt thereof, is provided in the form of a besylate salt.

84. The pharmaceutical composition of any one of claims 80-83, wherein the composition comprises:
from about 1 mg to about 5 mg of the besylate salt of the compound of Formula (I);
from about 5 mg to about 25 mg of an SGLT-2 inhibitor, or a pharmaceutically acceptable salt or solvate thereof, selected from the group consisting of: empagliflozin, canagliflozin, dapagliflozin, and ertugliflozin, or a pharmaceutically acceptable salt or solvate of any of the foregoing;
from about 1 mg to about 50 mg of a DPP-4 inhibitor, or a pharmaceutically acceptable salt thereof, selected from the group consisting of: sitagliptin, saxagliptin, linagliptin, and alogliptin, or a pharmaceutically acceptable salt of any of the foregoing; and
one or more pharmaceutical excipients.

85. A pharmaceutical combination comprising
(a) the compound of Formula (I), 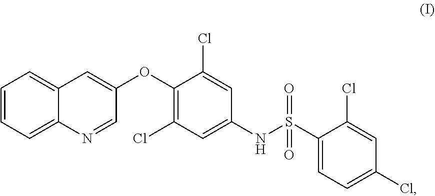 [SMILES: O=S(=O)(Nc1cc(Cl)c(Oc2cnc3ccccc3c2)c(Cl)c1)c1ccc(Cl)cc1Cl]
or a pharmaceutically acceptable salt thereof,
(b) an SGLT-2 inhibitor, or a pharmaceutically acceptable salt or solvate thereof, and
(c) a DPP-4 inhibitor, or a pharmaceutically acceptable salt thereof;
for concurrent or sequential administration for use in the treatment of non-alcoholic fatty liver disease (NAFLD).

86. The pharmaceutical combination of claim 85, wherein the SGLT-2 inhibitor, or a pharmaceutically acceptable salt or solvate thereof, is selected from the group consisting of: empagliflozin, canagliflozin, dapagliflozin, ertugliflozin, ipragliflozin, luseogliflozin, remogliflozin etabonate, serfliflozin etabonate, sotagliflozin, tofogliflozin, or a pharmaceutically acceptable salt or solvate of any of the foregoing.

87. The pharmaceutical combination of claim 85 or 86, wherein the DPP-4 inhibitor, or a pharmaceutically acceptable salt thereof, is selected from the group consisting of: sitagliptin, vildagliptin, saxagliptin, linagliptin, gemigliptin, anagliptin, teneligliptin, alogliptin, trelagliptin, omarigliptin, evogliptin, and dutogliptin, or a pharmaceutically acceptable salt of any of the foregoing.

88. The pharmaceutical combination of any one of claims 85-87, wherein the compound of Formula (I), or a pharmaceutically acceptable salt thereof, is provided in the form of a besylate salt.

89. The pharmaceutical combination of any one of claims 85-88, further comprising at least one pharmaceutically acceptable carrier.

90. A method of treating non-alcoholic fatty liver disease (NAFLD) in a subject in need thereof consisting essentially of administering to the subject
(a) the compound of Formula (I), 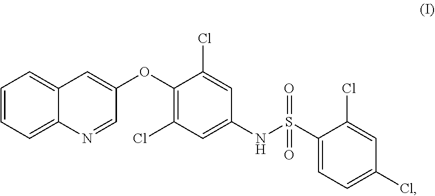 [SMILES: O=S(=O)(Nc1cc(Cl)c(Oc2cnc3ccccc3c2)c(Cl)c1)c1ccc(Cl)cc1Cl]
or a pharmaceutically acceptable salt thereof,
(b) an SGLT-2 inhibitor, or a pharmaceutically acceptable salt or solvate thereof, and
(c) a DPP-4 inhibitor, or a pharmaceutically acceptable salt thereof;
wherein the amounts of (a), (b), and (c) together are effective in treating NAFLD.

91. The method of claim 90, wherein the SGLT-2 inhibitor, or a pharmaceutically acceptable salt or solvate thereof, is selected from the group consisting of: empagliflozin, canagliflozin, dapagliflozin, ertugliflozin, ipragliflozin, luseogliflozin, remogliflozin etabonate, serfliflozin etabonate, sotagliflozin, tofogliflozin, or a pharmaceutically acceptable salt or solvate of any of the foregoing.

92. The method of claim 90 or 91, wherein the DPP-4 inhibitor, or a pharmaceutically acceptable salt thereof, is selected from the group consisting of: sitagliptin, vildagliptin, saxagliptin, linagliptin, gemigliptin, anagliptin, teneligliptin, alogliptin, trelagliptin, omarigliptin, evogliptin, and dutogliptin, or a pharmaceutically acceptable salt of any of the foregoing.

93. The method of any one of claims 90-92, wherein the compound of Formula (I), or a pharmaceutically acceptable salt thereof, is provided in the form of a besylate salt.
